SEQUENCE LISTING (6 sequences)

SEQ ID NO: 1 - DNA, 32 bp, artificial sequence
  Dbait32
acgcacgggtgttgggtcgtttgttcggatct

SEQ ID NO: 2 - DNA, 32 bp, artificial sequence
  Dbait32Ha
cgtaggtctgtttggtggctttgcagtggcac

SEQ ID NO: 3 - DNA, 32 bp, artificial sequence
  Dbait32Hb
gctaggcttgtttgctgggttgtaggcacagc

SEQ ID NO: 4 - DNA, 32 bp, artificial sequence
  Dbait32Hc
gctgtgcccacaacccagcaaacaagcctaga

SEQ ID NO: 5 - DNA, 32 bp, artificial sequence
  Dbait32Hd
gctaggtctgtttggtggctttgcagtggcac

SEQ ID NO: 6 - DNA, 32 bp, artificial sequence; identical to SEQ ID NO: 4
  Dbait molecule IIa
  misc_feature: at the 3′ end of the complementary strand the three last nucleotides with phosphorothioate or methylphosphonate backbone
  misc_feature (1)..(1): at the 5′ end, Lm = carboxamido oligoethylene glycol + C = single or double chain fatty acids, cholesterol, sugars, peptides or proteins
  misc_feature (1)..(1): at the 5′ end, Lm = tetraethyleneglycol + C = cholesterol
  stem_loop (1)..(32)
  modified_base (1)..(3): mod_base= phosphorothioate or methylphosphonate backbone
  misc_feature (32)..(32): loop L′ = 1,19-bis(phospho)-8-hydraza-2-hydroxy-4-oxa-9-oxo-nonadecane

(19) United States
(12) Patent Application Publication
(10) Pub. No.: US 2022/0143049 A1
(43) Pub. Date: May 12, 2022

(54) A DBAIT MOLECULE IN COMBINATION WITH KINASE INHIBITOR FOR THE TREATMENT OF CANCER

(71) Applicants: ONXEO, PARIS (FR); INSERM (INSTITUT NATIONAL DE LA SANTÉ ET DE LA RECHERCHE MÉDICALE), PARIS (FR); UNIVERSITE PAUL SABATIER TOULOUSE III, TOULOUSE CEDEX 9 (FR); INSTITUT CLAUDIUS REGAUD, TOULOUSE (FR)

(72) Inventors: FRANÇOISE BONO, TOULOUSE (FR); GILLES FAVRE, TOULOUSE CEDEX 1 (FR); OLIVIER CALVAYRAC, TOULOUSE CEDEX 1 (FR)

(21) Appl. No.: 17/593,474

(22) PCT Filed: Mar. 19, 2020
(86) PCT No.: PCT/EP2020/057555

§ 371 Date: Sep. 20, 2021

(30) Foreign Application Priority Data
Mar. 21, 2019 (EP) 19305349.3

(51) Int. Cl. A61K 31/6615; C12N 15/11; C12N 15/113; A61K 45/06; A61K 47/54

(52) U.S. Cl. CPC A61K 31/6615; C12N 15/111; C12N 15/113; A61K 45/06; C12N 2320/31; C12N 2310/13; C12N 2310/315; C12N 2310/3515; A61K 47/544

(57) ABSTRACT

The present invention relates to the combination of a Dbait molecule with a protein kinase inhibitor for treating cancer.

### FIELD OF THE INVENTION

The present invention relates to the field of medicine, in particular of oncology.

### BACKGROUND OF THE INVENTION

The emergence of diverse resistance mechanisms to targeted therapy is one of the foremost challenges in cancer today. Diverse drug-resistance mechanisms can arise from pre-existing mutations before treatment but more and more evidence support that small subpopulations of cancer cells can survive upon selective drug pressure. These surviving cells become Drug Tolerant Persisters (DTP), with little to-no population growth, for weeks to months, thus providing a latent reservoir of tumor cells. Twenty percent of DTPs undergo phenotypic transition to become Drug Tolerant Expended Persisters which resume their proliferation, and acquire genetic modifications of resistance (e.g. EGFR T790M) at the origin of tumor recurrence in patient. Cancer therapy has traditionally focused on eliminating fast-growing populations of cells and in that case, we are face to a new paradigm. The first evidence of the role of persisters or drug tolerant cells (DTP) in targeted therapies acquired resistance mechanisms was described by Sharma et al (Cell 2010, 141, 69-80) and further described in several publications (Hata et al. Nat Med 2016, 22(3): 262-269. doi:10.1038/nm.4040., Ramirez et al. Nat Comm 2016, DOI: 10.1038/ncomms10690, Guler et al. Can Cell 2017, 32, 221-237). These works demonstrated that drug-resistance mechanisms can emerge from persisters, derived from a single, recent ancestor cell and grown under the same selective pressure. This heterogeneity presents considerable clinical challenges for ‘personalized’ therapy: even if an effective therapy is selected for one PERC (persister-derived erlotinib-resistant colonies), there is no guarantee that this drug would be effective for other PERCs, which in practice may have been undetected. Persisters, which are a small subpopulation of the bulk cancer population, are difficult to study in a clinical setting, and there is no known molecular signature of having passed through this state clinically. However, Hata et al provide evidence that clinically relevant drug resistant cancer cells can both pre-exist and evolve from drug tolerant cells, and point persisters as a strategic target for new therapeutic opportunities to prevent or overcome resistance in the clinic.

Accordingly, new treatment methods are needed to successfully address these cells within cancer cell populations and the emergence of cancer cells resistant to therapies. Indeed, discovering new ways to eliminate the reservoir of DTP that fail to undergo cell death, preventing mutations occurring during the transition to DTEP, is of crucial importance to cure patients.

### SUMMARY OF THE INVENTION

The present invention provides a therapeutic agent DBait for the treatment of cancer in combination with kinase inhibitors, in particular in order to prevent or delay the apparition of acquired resistances to the kinase inhibitors. Indeed, the DBait molecule shows a targeted effect on persister cancer cells, thereby preventing or delaying the cancer relapse and/or preventing or delaying the apparition of acquired resistances to the kinase inhibitors.

Accordingly, the present invention relates to a pharmaceutical composition, a combination or a kit comprising a Dbait molecule and a protein kinase inhibitor. More specifically, the pharmaceutical composition, the combination or the kit comprises a Dbait molecule and one or several protein kinase inhibitors, targeting the same or different kinases.

In one aspect, the kinase inhibitor is an inhibitor targeting one or several targets selected in the list consisting of EGFR family, ALK, B-Raf, MEK, FGFR1, FGFR2, FGFR3, FGFR4, FLT3, IGF1R, c-Met, JAK family, PDGFR α and β, RET, AXL, c-KIT, TrkA, TrkB, TrkC, ROS1, BTK and Syk. For instance, the kinase inhibitor can be selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No 1421373-98-9), poziotinib, WZ4002, Crizotinib, entrectinib, ceritinib, alectinib, lorlatinib, TSR-011, CEP-37440, ensartinib, Vemurafenib, dabrafenib, regorafenib, PLX4720, Cobimetinib, Trametinib, Binimetinib, Selumetinib, PD-325901, CI-1040, PD035901, U0126, TAK-733, Lenvatinib, Debio-1347, dovitinib, BLU9931, Sorafenib, sunitinib, lestaurtinib, tandutinib, quizartinib, crenolanib, gilteritinib, ponatinib, ibrutinib, Linsitinib, NVP-AEW541, BMS-536924, AG-1024, GSK1838705A, BMS-754807, PQ 401, ZD3463, NT157, Picropodophyllin (PPP), Tivantinib, JNJ-38877605, PF-04217903, foretinib (GSK 1363089), Merestinib, Ruxolitinib, tofacitinib, oclacitinib, baricitinib, filgotinib, cerdulatinib, gandotinib, momelotinib, pacritinib, PF-04965842, upadacitinib, peficitinib, fedratinib, imatinib, pazopanib, Telatinib, bosutinib, nilotinib, cabozantinib, Bemcentinib, amuvatinib, gilteritinib (ASP2215), glesatinib (MGCD 265), SGI-7079, Larotrectinib, RXDX-102, altiratinib, LOXO-195, sitravatinib, TPX-0005, DS-6051b, fostamatinib, entospletinib and TAK-659.

In a particular aspect, the tyrosine kinase inhibitor is an inhibitor of a protein kinase selected from the group consisting of EGFR, ALK and B-Raf, in particular a protein kinase inhibitor selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No 1421373-98-9), poziotinib, WZ4002, Crizotinib, entrectinib, ceritinib, alectinib, lorlatinib, TSR-011, CEP-37440, ensartinib, Vemurafenib, dabrafenib, regorafenib and PLX4720.

In a very specific aspect, the protein kinase inhibitor is a EGFR inhibitor, in particular a EGFR inhibitor selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No 1421373-98-9), poziotinib and WZ4002.

In another very specific aspect, the protein kinase inhibitor is a ALK inhibitor, in particular a ALK inhibitor selected from the group consisting of crizotinib, entrectinib, ceritinib, alectinib, brigatinib, lorlatinib, TSR-011, CEP-37440 and ensartinib. In one aspect, the Dbait molecule has at least one free end and a DNA double stranded portion of 20-200 bp with less than 60% sequence identity to any gene in a human genome. More particularly, the Dbait molecule has one of the following formulae:

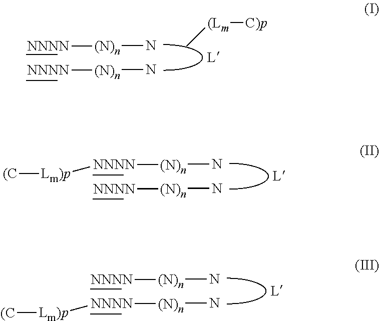

wherein N is a deoxynucleotide, n is an integer from 15 to 195, the underlined N refers to a nucleotide having or not a modified phosphodiester backbone, L′ is a linker, C is the molecule facilitating endocytosis selected from a lipophilic molecule or a ligand which targets cell receptor enabling receptor mediated endocytosis, L is a linker, m and p, independently, are an integer being 0 or 1.

Preferably, the Dbait molecule has the following formula:

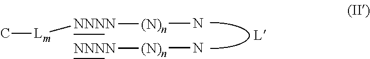

with the same definition than formulae (I), (II), and (III) for N, N, n, L, L′, C and m.

In a very specific aspect, the Dbait molecule has the following formula:

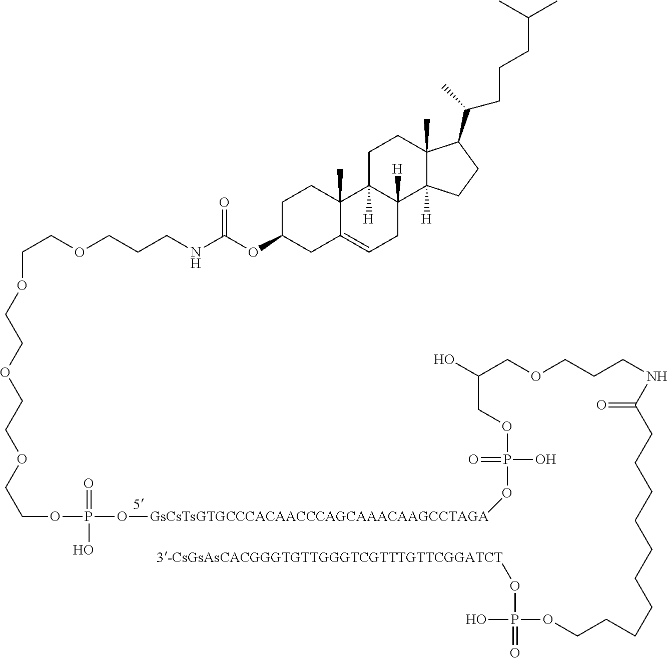

The present invention further relates to a pharmaceutical composition, a combination or the kit according to the present disclosure for use in the treatment of cancer. It also relates to a Dbait molecule as defined herein for use in the treatment of cancer in combination with a kinase inhibitor, in particular as defined herein. In addition, it relates to a Dbait molecule as defined herein for use in delaying and/or preventing development of a cancer resistant to a kinase inhibitor in a patient, in particular a kinase inhibitor as defined herein.

In one aspect, the cancer can be selected from the group consisting of leukemia, lymphoma, sarcoma, melanoma, and cancers of the head and neck, kidney, ovary, pancreas, prostate, thyroid, lung, esophagus, breast, bladder, brain, colorectum, liver, and cervix.

In a particular aspect, the cancer is selected from the group consisting of lung cancer, in particular non-small cell lung cancer, leukemia, in particular acute myeloid leukemia, chronic lymphocytic leukemia, lymphoma, in particular peripheral T-cell lymphoma, chronic myelogenous leukemia, squamous cell carcinoma of the head and neck, advanced melanoma with BRAF mutation, colorectal cancer, gastrointestinal stromal tumor, breast cancer, in particular HER2⁺ breast cancer, thyroid cancer, in particular advanced medullary thyroid cancer, kidney cancer, in particular renal cell carcinoma, prostate cancer, glioma, pancreatic cancer, in particular pancreatic neuroendocrine cancer, multiple myeloma, and liver cancer, in particular hepatocellular carcinoma. Finally, the present invention relates to a Dbait molecule as defined herein for use for a targeted effect against cancer persister cells in the treatment of cancer, in particular cancer persister cells to a kinase inhibitor as defined herein.

### BRIEF DESCRIPTION OF THE DRAWINGS

FIG. 1A: AsiDNA alone does not induce (EGFR)-addicted non-small cell lung cancer (NSCLC) cell lines PC9 and HCC827 cell death.

FIG. 1B: AsiDNA does not potentiate the efficacy of erlotinib on induced (EGFR)-addicted non-small cell lung cancer (NSCLC) cell lines PC9 and HCC827 cell death.

FIG. 1C: AsiDNA prevents the emergence of erlotinib-resistant clones.

FIG. 2: Long term efficacy of AsiDNA treatment on Erlotinib acquired resistance in (EGFR)-addicted non-small cell lung cancer (NSCLC) parental PC9 and subclones HCC827 sc2 and NSCLC PC9-3. AsiDNA treatment alone did not affect NSCLC cell survival (FIG. 2A-2C-2E). AsiDNA totally abrogated Erlotinib acquired resistance on the two subclones NSCLC HCC827 sc2 for 40 days (FIG. 2B) and NSCLC PC9-3 for 70 days (FIG. 2D) while it partially but significantly reduced resistance on NSCLC PC9 parental cell line (FIG. 2F).

FIG. 3: Long term efficacy of AsiDNA treatment on Osimertinib acquired resistance in (EGFR)-addicted non-small cell lung cancer (NSCLC) PC9-3. AsiDNA treatment alone did not affect cell survival (FIG. 3A). AsiDNA significantly reduced Osimertinib resistance on NSCLC PC9 parental cell line (FIG. 3B).

FIG. 4: Long term efficacy of AsiDNA treatment on Alectinib acquired resistance in (EGFR)-addicted non-small cell lung cancer (NSCLC) H3122. AsiDNA treatment alone did not affect cell survival (FIG. 4A). AsiDNA totally abrogated Alectinib acquired resistance on NSCLC H3122 cells for 40 days (FIG. 4B).

FIG. 5: AsiDNA in combination with Erlotinib significantly reduced the tumor growth in vivo. Erlotinib treatment alone transiently controls the tumor growth (FIG. 5B) and AsiDNA treatment alone slightly abrogates the tumor growth (FIG. 5C) in comparison with no treatment (FIG. 5A). AsiDNA in combination with Erlotinib significantly reduces the tumor growth and induces two complete regressions (FIG. 5D).

### DETAILED DESCRIPTION OF THE INVENTION

The present invention relates to the capacity of a Dbait molecule to strongly decrease the emergence of persistent cancer cells, in particular of cancer cells resistant to a kinase inhibitor.

Accordingly, the present invention relates to a pharmaceutical composition, a combination or a kit (kit-of-parts) comprising a Dbait molecule and a kinase inhibitor, in particular for use for treating cancer. More specifically, the pharmaceutical composition, the combination or the kit comprises a Dbait molecule and one or several protein kinase inhibitors, targeting the same or different kinases.

The present invention also relates to a pharmaceutical composition comprising a Dbait molecule and a kinase inhibitor for use in the treatment of a cancer; to a combination or a kit (kit-of-parts) comprising a Dbait molecule and a kinase inhibitor as a combined preparation for simultaneous, separate or sequential use, in particular for use in the treatment of cancer. It further relates to a method for treating a cancer in a subject in need thereof, comprising administering a therapeutically effective amount of a Dbait molecule and a therapeutically effective amount of a kinase inhibitor, and optionally a pharmaceutically acceptable carrier. It relates to the use of a Dbait molecule and a kinase inhibitor for the manufacture of a drug for treating a cancer.

The present invention relates to a Dbait molecule or a pharmaceutical composition comprising a Dbait molecule for use for the treatment of cancer in combination of a kinase inhibitor. More particularly, it relates to a Dbait molecule or a pharmaceutical composition comprising a Dbait molecule for use in delaying and/or preventing development of a cancer resistant to a kinase inhibitor in a patient. It relates to a Dbait molecule for use in extending the duration of response to a kinase inhibitor in the cancer treatment of a patient. It also relates to a method for delaying and/or preventing development of a cancer resistant to a kinase inhibitor in a patient and/or for extending the duration of response to a kinase inhibitor in the cancer treatment of a patient, comprising administering a therapeutically effective amount of a Dbait molecule and a therapeutically effective amount of a kinase inhibitor, and optionally a pharmaceutically acceptable carrier. It relates to the use of a Dbait molecule for the manufacture of a drug for treating a cancer in combination with a kinase inhibitor, for delaying and/or preventing development of a cancer resistant to a kinase inhibitor in a patient and/or for extending the duration of response to a kinase inhibitor in the cancer treatment of a patient.

Finally, more generally, the present invention relates to a Dbait molecule for use for inhibiting or preventing proliferation of cancer persistent cells or formation of colonies of cancer persistent cells, thereby preventing or delaying the cancer relapse and/and the emergence of acquired resistance to a cancer treatment. In addition, this effect against cancer persistent cells may allow to reach a complete response to the cancer treatment. Indeed, the Dbait molecule would be able to eliminate the cancer persistent cells. It also relates to a method for removing or decreasing the cancer persister cell population and/or for preventing or delaying the cancer relapse and/and the emergence of acquired resistance to a cancer treatment, comprising administering a therapeutically effective amount of a Dbait molecule, thereby removing or decreasing the cancer persister cell population. The Dbait treatment would be beneficial in targeting viable “persister” tumor cells and thus may prevent the emergence of drug-resistant clone(s), in particular in the context of a combined treatment with a kinase inhibitor.

### Definition

The terms “kit”, “product”, “combination” or “combined preparation”, as used herein, defines especially a “kit-of-parts” in the sense that the combination partners as defined above can be dosed independently or by use of different fixed combinations with distinguished amounts of the combination partners, i.e. simultaneously or at different time points. The parts of the kit-of-parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

Within the context of the invention, the term “treatment” denotes curative, symptomatic, preventive treatment as well as maintenance treatment. Pharmaceutical compositions, kits, products and combined preparations of the invention can be used in humans with existing cancer or tumor, including at early or late stages of progression of the cancer. The pharmaceutical compositions, kits, combinations, products and combined preparations of the invention will not necessarily cure the patient who has the cancer but will delay or slow the progression or prevent further progression of the disease, ameliorating thereby the patients' condition. In particular, the pharmaceutical compositions, kits, combinations, products and combined preparations of the invention reduce the development of tumors, reduce tumor burden, produce tumor regression in a mammalian host and/or prevent metastasis occurrence and cancer relapse. The pharmaceutical compositions, kits, combinations, products and combined preparations according to the present invention advantageously prevent, delay the emergence or the development of, decrease or remove the persister tumor cells and/or drug-tolerant expanded persisters.

By “therapeutically effective amount” it is meant the quantity of the compound of interest of the pharmaceutical composition, kit, combination, product or combined preparation of the invention which prevents, removes or reduces the deleterious effects of cancer in mammals, including humans, alone or in combination with the other active ingredients of the pharmaceutical composition, kit, combination, product or combined preparation. It is understood that the administered dose may be lower for each compound in the composition to the “therapeutically effective amount” define for each compound used alone or in combination with other treatments than the combination described here. The “therapeutically effective amount” of the composition will be adapted by those skilled in the art according to the patient, the pathology, the mode of administration, etc.

Whenever within this whole specification the terms “treatment of a cancer” or “treating a cancer” or the like are mentioned with reference to the pharmaceutical composition, kit, combination, product or combined preparation of the invention, there is meant: a) a method for treating a cancer, said method comprising administering a pharmaceutical composition, kit, combination, product or combined preparation of the invention to a patient in need of such treatment; b) the use of a pharmaceutical composition, kit, combination, product or combined preparation of the invention for the treatment of a cancer; c) the use of a pharmaceutical composition, kit, combination, product or combined preparation of the invention for the manufacture of a medicament for the treatment of a cancer; and/or d) a pharmaceutical composition, kit, combination, product or combined preparation of the invention for use in the treatment a cancer.

The pharmaceutical compositions, kits, combinations, products or combined preparations contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition, kit, combination, product or combined preparation can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient(s); in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavouring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions, kits, combinations, products or combined preparations are advantageously applied by injection or intravenous infusion of suitable sterile solutions or as oral dosage by the digestive tract. Methods for the safe and effective administration of most of these therapeutic agents are known to those skilled in the art. In addition, their administration is described in the standard literature.

By “persister cell”, “persister cancer cell”, “drug tolerant persister” or “DTP” is intended to refer to a small subpopulation of cancer cells that maintain viability under anti-cancer targeted therapy treatments, in particular a treatment with a kinase inhibitor. More particularly, it refers to cancer cells that have a tolerance to high concentrations of a treatment of a kinase inhibitor, when it is used in concentrations that are 100 of times higher than IC50. These cells have a slow growth and are almost quiescent.

The term “drug-tolerant expanded persister” or “DTEP” as used herein, refers to cancer cells that are capable to proliferate with continuous cancer drug treatment in high concentrations, in particular a treatment with a kinase inhibitor.

Dbait Molecules

The term “Dbait molecule” also known as signal interfering DNA (siDNA) as used herein, refers to a nucleic acid molecule, preferably a hairpin nucleic acid molecule, designed to counteract DNA repair. A Dbait molecule has at least one free end and a DNA double stranded portion of 20-200 bp with less than 60% sequence identity to any gene in a human genome.

Preferably, the Dbait molecules for use in the present invention, conjugated or not, can be described by the following formulae:

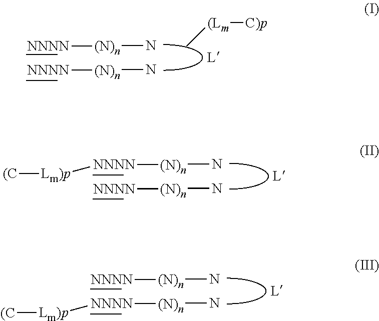

wherein N is a deoxynucleotide, n is an integer from 15 to 195, the underlined N refers to a nucleotide having or not a modified phosphodiester backbone, L′ is a linker, C is a molecule facilitating endocytosis preferably selected from a lipophilic molecule and a ligand which targets cell receptor enabling receptor mediated endocytosis, L is a linker, m and p, independently, are an integer being 0 or 1.

In preferred embodiments, the Dbait molecules of formulae (I), (II), or (III) have one or several of the following features:
N is a deoxynucleotide, preferably selected from the group consisting of A (adenine), C (cytosine), T (thymine) and G (guanine) and selected so as to avoid occurrence of a CpG dinucleotide and to have less than 80% or 70%, even less than 60% or 50% sequence identity to any gene in a human genome; and/or,
n is an integer from 15 to 195, from 19-95, from 21 to 95, from 27 to 95, from 15 to 45, from 19 to 45, from 21 to 45, or from 27 to 45; preferably n is 27; and/or,
the underlined N refers to a nucleotide having or not a phosphorothioate or methylphosphonate backbone, more preferably a phosphorothioate backbone; preferably, the underlined N refers to a nucleotide having a modified phosphodiester backbone; and/or,
the linker L′ is selected from the group consisting of hexaethyleneglycol, tetradeoxythymidylate (T4), 1,19-bis(phospho)-8-hydraza-2-hydroxy-4-oxa-9-oxo-nonadecane; and 2,19-bis(phosphor)-8-hydraza-1-hydroxy-4-oxa-9-oxo-nonadecane; and/or,
m is 1 and L is a carboxamido polyethylene glycol, more preferably carboxamido triethylene or tetraethylene glycol; and/or,
C is selected from the group consisting of a cholesterol, single or double chain fatty acids such as octadecyl, oleic acid, dioleoyl or stearic acid, or ligand (including peptide, protein, aptamer) which targets cell receptor such as folic acid, tocopherol, sugar such as galactose and mannose and their oligosaccharide, peptide such as RGD and bombesin, and protein such transferring and integrin, preferably is a cholesterol or a tocopherol, still more preferably a cholesterol.

Preferably, C-Lm is a triethyleneglycol linker (10-O-[1-propyl-3-N-carbamoylcholesteryl]-triethyleneglycol radical. Alternatively, C-Lm is a tetraethyleneglycol linker (10-O-[1-propyl-3-N-carbamoylcholesteryl]-tetraethyleneglycol radical.

In a preferred embodiment, the Dbait molecule has the following formula:

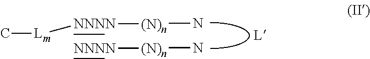

with the same definition than formulae (I), (II), and (III) for N, N, n, L, L′, C and m.

In a particular embodiment, the Dbait molecules are those extensively described in PCT patent applications WO2005/040378, WO2008/034866, WO2008/084087 and WO2011/161075, the disclosure of which is incorporated herein by reference.

Dbait molecules may be defined by a number of characteristics necessary for their therapeutic activity, such as their minimal length, the presence of at least one free end, and the presence of a double stranded portion, preferably a DNA double stranded portion. As will be discussed below, it is important to note that the precise nucleotide sequence of Dbait molecules does not impact on their activity. Furthermore, Dbait molecules may contain a modified and/or non-natural backbone.

Preferably, Dbait molecules are of non-human origin (i.e., their nucleotide sequence and/or conformation (e.g., hairpin) does not exist as such in a human cell), most preferably of synthetic origin. As the sequence of the Dbait molecules plays little, if any, role, Dbait molecules have preferably no significant degree of sequence homology or identity to known genes, promoters, enhancers, 5′- or 3′-upstream sequences, exons, introns, and the like. In other words, Dbait molecules have less than 80% or 70%, even less than 60% or 50% sequence identity to any gene in a human genome. Methods of determining sequence identity are well known in the art and include, e.g., Blast. Dbait molecules do not hybridize, under stringent conditions, with human genomic DNA. Typical stringent conditions are such that they allow the discrimination of fully complementary nucleic acids from partially complementary nucleic acids.

In addition, the sequence of the Dbait molecules is preferably devoid of CpG in order to avoid the well-known toll-like receptor-mediated immunological reactions.

The length of Dbait molecules may be variable, as long as it is sufficient to allow appropriate binding of Ku protein complex comprising Ku and DNA-PKcs proteins. It has been showed that the length of Dbait molecules must be greater than 20 bp, preferably about 32 bp, to ensure binding to such a Ku complex and allowing DNA-PKcs activation. Preferably, Dbait molecules comprise between 20-200 bp, more preferably 24-100 bp, still more preferably 26-100, and most preferably between 24-200, 25-200, 26-200, 27-200, 28-200, 30-200, 32-200, 24-100, 25-100, 26-100, 27-100, 28-100, 30-100, 32-200 or 32-100 bp. For instance, Dbait molecules comprise between 24-160, 26-150, 28-140, 28-200, 30-120, 32-200 or 32-100 bp. By “bp” is intended that the molecule comprise a double stranded portion of the indicated length. In a particular embodiment, the Dbait molecules having a double stranded portion of at least 32 pb, or of about 32 bp, comprise the same nucleotide sequence than Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5). Optionally, the Dbait molecules have the same nucleotide composition than Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) but their nucleotide sequence is different. Then, the Dbait molecules comprise one strand of the double stranded portion with 3 A, 6 C, 12 G and 11 T. Preferably, the sequence of the Dbait molecules does not contain any CpG dinucleotide.

Alternatively, the double stranded portion comprises at least 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5). In a more particular embodiment, the double stranded portion consists in 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5).

The Dbait molecules as disclosed herein must have at least one free end, as a mimic of double strand breaks (DSB). Said free end may be either a free blunt end or a 573T-protruding end. The “free end” refers herein to a nucleic acid molecule, in particular a double-stranded nucleic acid portion, having both a 5′ end and a 3′ end or having either a 3′end or a 5′ end. Optionally, one of the 5′ and 3′ end can be used to conjugate the nucleic acid molecule or can be linked to a blocking group, for instance a or 3′-3′nucleotide linkage.

In a particular embodiment, they contain only one free end. Preferably, Dbait molecules are made of hairpin nucleic acids with a double-stranded DNA stem and a loop. The loop can be a nucleic acid, or other chemical groups known by skilled person or a mixture thereof. A nucleotide linker may include from 2 to 10 nucleotides, preferably, 3, 4 or 5 nucleotides. Non-nucleotide linkers non-exhaustively include abasic nucleotide, polyether, polyamine, polyamide, peptide, carbohydrate, lipid, polyhydrocarbon, or other polymeric compounds (e.g. oligoethylene glycols such as those having between 2 and 10 ethylene glycol units, preferably 3, 4, 5, 6, 7 or 8 ethylene glycol units). A preferred linker is selected from the group consisting of hexaethyleneglycol, tetradeoxythymidylate (T4) and other linkers such as 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane. Accordingly, in a particular embodiment, the Dbait molecules can be a hairpin molecule having a double stranded portion or stem comprising at least 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) and a loop being a hexaethyleneglycol linker, a tetradeoxythymidylate linker (T4) 1,19-bis(phospho)-8-hydraza-2-hydroxy-4-oxa-9-oxo-nonadecane or 2,19-bis(phosphor)-8-hydraza-1-hydroxy-4-oxa-9-oxo-nonadecane. In a more particular embodiment, those Dbait molecules can have a double stranded portion consisting in 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5).

Dbait molecules preferably comprise a 2′-deoxynucleotide backbone, and optionally comprise one or several (2, 3, 4, 5 or 6) modified nucleotides and/or nucleobases other than adenine, cytosine, guanine and thymine. Accordingly, the Dbait molecules are essentially a DNA structure. In particular, the double-strand portion or stem of the Dbait molecules is made of deoxyribonucleotides.

Preferred Dbait molecules comprise one or several chemically modified nucleotide(s) or group(s) at the end of one or of each strand, in particular in order to protect them from degradation. In a particular preferred embodiment, the free end(s) of the Dbait molecules is(are) protected by one, two or three modified phosphodiester backbones at the end of one or of each strand. Preferred chemical groups, in particular the modified phosphodiester backbone, comprise phosphorothioates. Alternatively, preferred Dbait have 3′-3′ nucleotide linkage, or nucleotides with methylphosphonate backbone. Other modified backbones are well known in the art and comprise phosphoramidates, morpholino nucleic acid, 2′-0,4′-C methylene/ethylene bridged locked nucleic acid, peptide nucleic acid (PNA), and short chain alkyl, or cycloalkyl intersugar linkages or short chain heteroatomic or heterocyclic intrasugar linkages of variable length, or any modified nucleotides known by skilled person. In a first preferred embodiment, the Dbait molecules have the free end(s) protected by one, two or three modified phosphodiester backbones at the end of one or of each strand, more preferably by three modified phosphodiester backbones (in particular phosphorothioate or methylphosphonate) at least at the 3′end, but still more preferably at both 5′ and 3′ ends.

In a most preferred embodiment, the Dbait molecule is a hairpin nucleic acid molecule comprising a DNA double-stranded portion or stem of 32 bp (e.g., with a sequence selected from the group consisting of SEQ ID Nos 1-5, in particular SEQ ID No 4) and a loop linking the two strands of the DNA double-stranded portion or stem comprising or consisting of a linker selected from the group consisting of hexaethyleneglycol, tetradeoxythymidylate (T4) and 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane, the free ends of the DNA double-stranded portion or stem (i.e. at the opposite of the loop) having three modified phosphodiester backbones (in particular phosphorothioate internucleotidic links).

Said nucleic acid molecules are made by chemical synthesis, semi-biosynthesis or biosynthesis, any method of amplification, followed by any extraction and preparation methods and any chemical modification. Linkers are provided so as to be incorporable by standard nucleic acid chemical synthesis. More preferably, nucleic acid molecules are manufactured by specially designed convergent synthesis: two complementary strands are prepared by standard nucleic acid chemical synthesis with the incorporation of appropriate linker precursor, after their purification, they are covalently coupled together.

Optionally, the nucleic acid molecules may be conjugated to molecules facilitating endocytosis or cellular uptake.

In particular, the molecules facilitating endocytosis or cellular uptake may be lipophilic molecules such as cholesterol, single or double chain fatty acids, or ligands which target cell receptor enabling receptor mediated endocytosis, such as folic acid and folate derivatives or transferrin (Goldstein et al. Ann. Rev. Cell Biol. 1985 1:1-39; Leamon & Lowe, Proc Natl Acad Sci USA. 1991, 88: 5572-5576.). The molecule may also be tocopherol, sugar such as galactose and mannose and their oligosaccharide, peptide such as RGD and bombesin and protein such as integrin. Fatty acids may be saturated or unsaturated and be in C₄-C₂₈, preferably in C₁₄-C₂₂, still more preferably being in C₁₈ such as oleic acid or stearic acid. In particular, fatty acids may be octadecyl or dioleoyl. Fatty acids may be found as double chain form linked with in appropriate linker such as a glycerol, a phosphatidylcholine or ethanolamine and the like or linked together by the linkers used to attach on the Dbait molecule. As used herein, the term “folate” is meant to refer to folate and folate derivatives, including pteroic acid derivatives and analogs. The analogs and derivatives of folic acid suitable for use in the present invention include, but are not limited to, antifolates, dihydrofolates, tetrahydrofolates, folinic acid, pteropolyglutamic acid, 1-deza, 3-deaza, 5-deaza, 8-deaza, 10-deaza, 1,5-deaza, 5,10 dideaza, 8,10-dideaza, and 5,8-dideaza folates, antifolates, and pteroic acid derivatives. Additional folate analogs are described in US2004/242582. Accordingly, the molecule facilitating endocytosis may be selected from the group consisting of single or double chain fatty acids, folates and cholesterol. More preferably, the molecule facilitating endocytosis is selected from the group consisting of dioleoyl, octadecyl, folic acid, and cholesterol. In a most preferred embodiment, the nucleic acid molecule is conjugated to a cholesterol.

The Dbait molecules facilitating endocytosis may be conjugated to molecules facilitating endocytosis, preferably through a linker. Any linker known in the art may be used to attach the molecule facilitating endocytosis to Dbait molecules. For instance, WO09/126933 provides a broad review of convenient linkers pages 38-45. The linker can be non-exhaustively, aliphatic chain, polyether, polyamine, polyamide, peptide, carbohydrate, lipid, polyhydrocarbon, or other polymeric compounds (e.g. oligoethylene glycols such as those having between 2 and 10 ethylene glycol units, preferably 3, 4, 5, 6, 7 or 8 ethylene glycol units, still more preferably 3 ethylene glycol units), as well as incorporating any bonds that may be break down by chemical or enzymatical way, such as a disulfide linkage, a protected disulfide linkage, an acid labile linkage (e.g., hydrazone linkage), an ester linkage, an ortho ester linkage, a phosphonamide linkage, a biocleavable peptide linkage, an azo linkage or an aldehyde linkage. Such cleavable linkers are detailed in WO2007/040469 pages 12-14, in WO2008/022309 pages 22-28.

In a particular embodiment, the nucleic acid molecule can be linked to one molecule facilitating endocytosis. Alternatively, several molecules facilitating endocytosis (e.g., two, three or four) can be attached to one nucleic acid molecule.

In a specific embodiment, the linker between the molecule facilitating endocytosis, in particular cholesterol, and nucleic acid molecule is CO—NH—(CH₂—CH₂—O)ₙ, wherein n is an integer from 1 to 10, preferably n being selected from the group consisting of 3, 4, 5 and 6. In a very particular embodiment, the linker is CO—NH—(CH₂—CH₂—O)₄ (carboxamido tetraethylene glycol) or CO—NH—(CH₂—CH₂—O)₃ (carboxamido triethylene glycol). The linker can be linked to nucleic acid molecules at any convenient position which does not modify the activity of the nucleic acid molecules. In particular, the linker can be linked at the 5′ end. Therefore, in a preferred embodiment, the contemplated conjugated Dbait molecule is a Dbait molecule having a hairpin structure and being conjugated to the molecule facilitating endocytosis, preferably through a linker, at its 5′ end.

In another specific embodiment, the linker between the molecule facilitating endocytosis, in particular cholesterol, and nucleic acid molecule is dialkyl-disulfide {e.g., (CH₂)ᵣ—S—S—(CH₂)ₛ with r and s being integer from 1 to 10, preferably from 3 to 8, for instance 6}.

In a most preferred embodiment, the conjugated Dbait molecule is a hairpin nucleic acid molecule comprising a DNA double-stranded portion or stem of 32 bp and a loop linking the two strands of the DNA double-stranded portion or stem comprising or consisting of a linker selected from the group consisting of hexaethyleneglycol, tetradeoxythymidylate (T4), 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane, the free ends of the DNA double-stranded portion or stem (i.e. at the opposite of the loop) having three modified phosphodiester backbones (in particular phosphorothioate internucleotidic links) and said Dbait molecule being conjugated to a cholesterol at its 5′ end, preferably through a linker (e.g. carboxamido oligoethylene glycol, preferably carboxamido triethylene or tetraethylene glycol).

In a particular embodiment, the Dbait molecules can be conjugated Dbait molecules such as those extensively described in PCT patent application WO2011/161075, the disclosure of which is incorporated herein by reference.

In a preferred embodiment, NNNN—(N)ₙ—N comprises at least 6, 8, 10, 12, 14, 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) or consists in 20, 22, 24, 26, 28, 30 or 32 consecutive nucleotides of Dbait32, Dbait32Ha, Dbait32Hb, Dbait32Hc or Dbait32Hd. In a particular embodiment, NNNN—(N)ₙ—N comprises or consists in Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5), more preferably Dbait32Hc (SEQ ID NO: 4).

According, the conjugated Dbait molecules may be selected from the group consisting of:

with NNNN—(N)ₙ—N being SEQ ID NO: 1;

with NNNN—(N)ₙ—N being SEQ ID NO: 2;

with NNNN—(N)ₙ—N being SEQ ID NO: 3;

with NNNN—(N)ₙ—N being SEQ ID NO: 4; or

with NNNN—(N)ₙ—N being SEQ ID NO: 5

In one preferred embodiment, the Dbait molecule has the following formula:

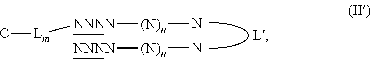

wherein
NNNN—(N)ₙ—N comprises 28, 30 or 32 nucleotides, preferably 32 nucleotides; and/or
the underlined nucleotide refers to a nucleotide having or not a phosphorothioate or methylphosphonate backbone, more preferably a phosphorothioate backbone; preferably, the underlined nucleotide refers to a nucleotide having a phosphorothioate or methylphosphonate backbone, more preferably a phosphorothioate backbone; and/or,
the linker L′ is selected from the group consisting of hexaethyleneglycol, tetradeoxythymidylate (T4), 1,19-bis(phospho)-8-hydraza-2-hydroxy-4-oxa-9-oxo-nonadecane or 2,19-bis(phosphor)-8-hydraza-1-hydroxy-4-oxa-9-oxo-nonadecane; and/or,
m is 1 and L is a carboxamido polyethylene glycol, more preferably carboxamido triethylene or tetraethylene glycol; and/or,
C is selected from the group consisting of a cholesterol, single or double chain fatty acids such as octadecyl, oleic acid, dioleoyl or stearic acid, or ligand (including peptide, protein, aptamer) which targets cell receptor such as folic acid, tocopherol, sugar such as galactose and mannose and their oligosaccharide, peptide such as RGD and bombesin, and protein such transferring and integrin, preferably is a cholesterol.

In a very specific embodiment, the Dbait molecule (also referred herein as AsiDNA) has the following formula:

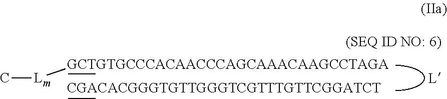

wherein C is a cholesteryl, Lm is a tetraethylene glycol, and L′ is 1,19-bis(phospho)-8-hydraza-2-hydroxy-4-oxa-9-oxo-nonadecane; also represented by the following formula:

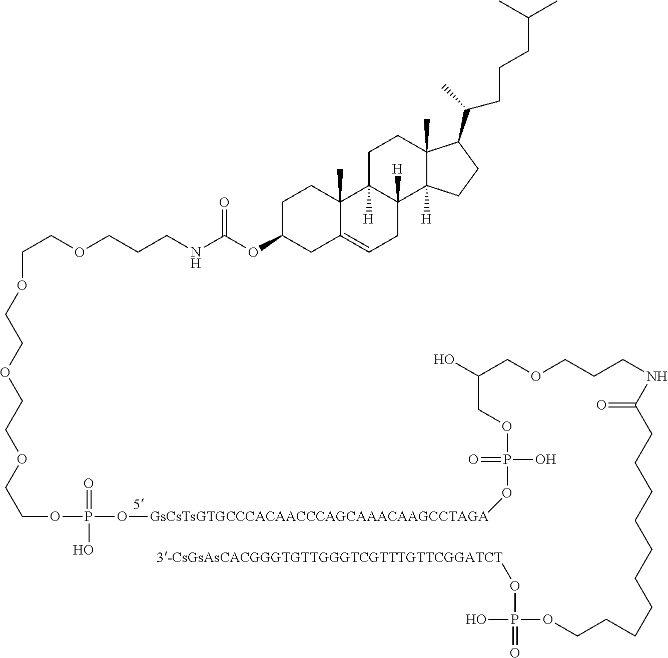

“s” refers to a phosphorothioate link between two nucleotides.

Kinase Inhibitors

The kinase inhibitor of the present invention is a kinase inhibitor for treating cancer. In particular, the kinase can be a tyrosine kinase, a serine/threonine kinase or a kinase with dual specificity. In a particular aspect, the kinase inhibitor is known to be associated with an acquired resistance during the cancer treatment. In a very particular aspect, the kinase inhibitor is associated with the occurrence of persister cancer cells during a treatment of cancer with this kinase inhibitor.

The kinase inhibitors may target any one of the following kinases: EGFR family, ALK, B-Raf, MEK, FGFR1, FGFR2, FGFR3, FGFR4, FLT3, IGF1R, c-Met, JAK family, PDGFR α and β, RET, AXL, c-KIT, TrkA, TrkB, TrkC, ROS1, BTK and Syk.

In one aspect, the kinase inhibitor is an inhibitor targeting a receptor tyrosine kinase, especially one selected from the group consisting of EGFR family, ALK, FGFR1, FGFR2, FGFR3, FGFR4, c-Met, RET, IGF1R, PDGFR α and β, c-KIT, FLT3, AXL, TrkA, TrkB, TrkC, and ROS1.

In a particular aspect, the kinase inhibitor is an inhibitor targeting a tyrosine kinase selected from the group consisting of EGFR, ALK, B-Raf, MEK, c-Met, JAK, PDGFR α and β, RET and BTK. For instance, a group of tyrosine kinases evolutionary and structurally related to ALK is RET, ROS1, AXL and Trk families kinases.

The kinase inhibitor is a small organic molecule. The term excludes biological macromolecules (e.g.; proteins, nucleic acids, etc.). Preferred small organic molecules range in size up to 2000 Da, and most preferably up to about 1000 Da.

The kinase inhibitor may target EGFR (epidermal growth factor receptor), also called ErbB-1 and HER1 (see UniprotKB—P00533). The EGFR kinase inhibitors are well-known. For instance, reviews are published disclosing such EGFR kinase inhibitors (Expert Opinion on Therapeutic Patents December 2002, Vol. 12, No. 12, Pages 1903-1907; Kane, Expert Opinion on Therapeutic Patents February 2006, Vol. 16, No. 2, Pages 147-164; Traxler, Expert Opinion on Therapeutic Patents December 1998, Vol. 8, No. 12, Pages 1599-1625; Singh et al, Mini Rev Med Chem. 2016; 16(14):1134-66; Cheng et al, Curr Med Chem. 2016; 23(29):3343-3359; Milik et al, Eur J Med Chem. 2017 Dec. 15; 142:131-151.; Murtuza et al, Cancer Res. 2019 Feb. 15; 79(4):689-698; Tan et al, Onco Targets Ther. 2019 Jan. 18; 12:635-645; Roskoski, Pharmacol Res. 2019 January; 139:395-411; Mountzios, Ann Transl Med. 2018 April; 6(8):140; Tan et al, Mol Cancer. 2018 Feb. 19; 17(1):29), the disclosure of which being incorporated herein by reference. Patent applications also disclose EGFR kinase inhibitors, for instance and non-exhaustively WO19010295, WO19034075, WO18129645, WO18108064, WO18050052, WO18121758, WO18218963, WO17114383, WO17049992, WO17008761, WO17015363, WO17016463, WO17117680, WO17205459, WO16112847, WO16054987, WO16070816, WO16079763, WO16125186, WO16123706, WO16050165, WO15081822, WO12167415, WO13138495, WO10129053, WO10076764, WO09143389, WO05065687, WO05018677, WO05027972, WO04011461, WO0134574, the disclosure of which being incorporated herein by reference. Specific examples of EGFR kinase inhibitors are disclosed in the following table.

The kinase inhibitors may target ALK (Anaplastic lymphoma kinase, also known as ALK tyrosine kinase receptor or CD246; UniprotKB—Q9UM73). The ALK kinase inhibitors are well-known. For instance, reviews are published disclosing such ALK kinase inhibitors (Beardslee et al, J Adv Pract Oncol. 2018 January-February; 9(1):94-101; Pacenta et al, Drug Des Devel Ther. 2018 Oct. 23; 12:3549-3561; Spagnuolo et al, Expert Opin Emerg Drugs. 2018 September; 23(3):231-241; Peters et al, Curr Treat Options Oncol. 2018 May 28; 19(7):37; Goldings et al, Mol Cancer. 2018 Feb. 19; 17(1):52; Karachaliou et al, Expert Opin Investig Drugs. 2017 June; 26(6):713-722; Liu et al, Curr Med Chem. 2017; 24(6):590-613; Crescenzo et al, Curr Opin Pharmacol. 2015 August; 23:39-44; Sgambato et al, Expert Rev Anticancer Ther. 2018 January; 18(1):71-80; Michellys et al, Bioorg Med Chem Lett. 2016 Feb. 1; 26(3):1090-1096; Straughan et al, Curr Drug Targets. 2016; 17(6):739-45), the disclosure of which being incorporated herein by reference. Patent applications also disclose ALK kinase inhibitors, for instance and non-exhaustively WO04080980, WO05016894, WO05009389, WO09117097, WO09143389, WO09132202, WO10085597, WO10143664, WO11138751, WO12037155, WO12017239, WO12023597, WO13013308, WO14193932, WO15031666, WO15127629, WO15180685, WO15194764, WO17076355, WO18001251, WO18044767, WO18094134, WO18127184, the disclosure of which being incorporated herein by reference. Specific examples of ALK kinase inhibitors are disclosed in the following table.

The kinase inhibitors may target B-Raf (Serine/threonine-protein kinase B-raf, also known as Proto-oncogene B-Raf, p94 or v-Raf murine sarcoma viral oncogene homolog B1; UniprotKB—P15056). The B-Raf kinase inhibitors are well-known. For instance, reviews are published disclosing such B-Raf kinase inhibitors (Tsai et al, PNAS Feb. 26, 2008 105 (8) 3041-3046, Garnett et Marais, 2004 Cancer cell, Volume 6, Issue 4, Pages 313-319; Wilmott et al 2012, Cancer Therapy: Clinical, Volume 18, Issue 5; Fujimura et al, Expert Opin Investig Drugs. 2019 February; 28(2):143-148, Trojaniello et al, Expert Rev Clin Pharmacol. 2019 March; 12(3):259-266; Kakadia et al, Onco Targets Ther. 2018 Oct. 17; 11:7095-7107; Roskoski, Pharmacol Res. 2018 September; 135:239-258; Eroglu et al, Ther Adv Med Oncol. 2016 January; 8(1):48-56), the disclosure of which being incorporated herein by reference. Patent applications also disclose B-Raf kinase inhibitors, for instance and non-exhaustively WO14164648, WO14164648, WO14206343, WO13040515, WO11147764, WO11047238, WO11025968, WO11025951, WO11025938, WO11025965, WO11090738, WO09143389, WO09111280, WO09111279, WO09111278, WO09111277, WO08068507, WO08020203, WO07119055, WO07113558, WO07071963, WO07113557, WO06079791, WO06067446, WO06040568, WO06024836, WO06024834, WO06003378, WO05123696, the disclosure of which being incorporated herein by reference. Specific examples of B-Raf kinase inhibitors are disclosed in the following table.

The kinase inhibitors may target MEK (Mitogen-activated protein kinase kinase, also known as MAPKK, MP2K, MAPKK, MAPK/ERK kinase, JNK-activating kinase, c-Jun N-terminal kinase kinase (JNKK), Stress-activated protein kinase kinase (SAPKK); UniprotKB—Q02750 (MP2K1), P36507 (MP2K2), P46734 (MP2K3), P45985 (MP2K4), 013163 (MP2K5), P52564 (MP2K6), 014733 (MP2K7)). Preferably, the kinase inhibitors target MEK-1 (also known as MAP2K1, MP2K1, MAPKK 1 or MKK1) and/or MEK-2 (also known as MAP2K2, MP2K2, MAPKK 2 or MKK2). Both MEK-1 and MEK-2 function specifically in the MAPK/ERK cascade. The MEK kinase inhibitors are well-known. For instance, reviews are published disclosing such MEK kinase inhibitors (Kakadia et al, Onco Targets Ther. 2018 Oct. 17; 11:7095-7107; Steeb et al, Eur J Cancer. 2018 November; 103:41-51; Sarkisian and Davar, Drug Des Devel Ther. 2018 Aug. 20; 12:2553-2565; Roskoski, Pharmacol Res. 2018 September; 135:239-258; Eroglu et al, Ther Adv Med Oncol. 2016 January; 8(1):48-56), the disclosure of which being incorporated herein by reference. Patent applications also disclose MEK kinase inhibitors, for instance and non-exhaustively WO15022662, WO15058589, WO14009319, WO14204263, WO13107283, WO13136249, WO13136254, WO12095505, WO12059041, WO11047238, WO11047055, WO11054828, WO10017051, WO10108652, WO10121646, WO10145197, WO09129246, WO09018238, WO09153554, WO09018233, WO09013462, WO09093008, WO08089459, WO07014011, WO07044515, WO07071951, WO07022529, WO07044084, WO07088345, WO07121481, WO07123936, WO06011466, WO06011466, WO06056427, WO06058752, WO06133417, WO05023251, WO05028426, WO05051906, WO05051300, WO05051301, WO05051302, WO05023759, WO04005284, WO03077855, WO03077914, WO02069960, WO0168619, WO0176570, WO0041994, WO0042022, WO0042003, WO0042002, WO0056706, WO0068201, WO9901426, the disclosure of which being incorporated herein by reference. Specific examples of MEK kinase inhibitors are disclosed in the following table.

The kinase inhibitors may target FGFR (Fibroblast growth factor receptor; UniprotKB—P11362 (FGFR1), P21802 (FGFR2), P22607 (FGFR3), P22455 (FGFR4)). The FGFR kinase inhibitors are well-known. For instance, reviews are published disclosing such FGFR kinase inhibitors (Katoh, Int J Mol Med. 2016 July; 38(1):3-15; Rizvi et Borad, J Gastrointest Oncol. 2016 October; 7(5):789-796; Tan et al, Onco Targets Ther. 2019 Jan. 18; 12:635-645, Shen et al, J Hematol Oncol. 2018 Sep. 19; 11(1):120; Porta et al, Crit Rev Oncol Hematol. 2017 May; 113:256-267; Cheng et al, Eur J Med Chem. 2017 Jan. 27; 126:476-490), the disclosure of which being incorporated herein by reference. Patent applications also disclose FGFR kinase inhibitors, for instance and non-exhaustively WO19034075, WO19034076, WO19001419, WO18028438, WO18049781, WO18121650, WO18153373, WO18010514, WO17028816, WO17070708, WO16091849, WO16134320, WO16054483, WO15059668, WO14007951, WO14026125, WO14129477, WO14162039, WO14172644, WO13108809, WO13129369, WO13144339, WO13179033, WO13053983, WO12008563, WO12008564, WO12047699, WO09153592, WO08078091, WO08075068, WO06112479, WO04056822, the disclosure of which being incorporated herein by reference. Specific examples of FGFR kinase inhibitors are disclosed in the following table. The FGFR kinase inhibitor can be selective one or several FGFR family members, especially members selected from FGFR1, FGFR2, FGFR3 and FGFR4.

The kinase inhibitors may target FLT3 (Receptor-type tyrosine-protein kinase FLT3, also known as FL cytokine receptor, Fetal liver kinase-2 (FLK-2), Fms-like tyrosine kinase 3 (FLT-3), Stem cell tyrosine kinase 1 (STK-1) or CD antigen: CD135; UniprotKB—P36888). The FLT3 kinase inhibitors are well-known. For instance, reviews are published disclosing such FLT3 kinase inhibitors (Stone, Best Pract Res Clin Haematol. 2018 December; 31(4):401-404; Wu et al, J Hematol Oncol. 2018 Dec. 4; 11(1):133; Short et al, Ther Adv Hematol. 2019 Feb. 15; 10:2040620719827310; Elshoury et al, Expert Rev Anticancer Ther. 2019 March; 19(3):273-286; Zhi et al, Eur J Med Chem. 2018 Jul. 15; 155:303-315; Tiong I S, Wei A H, Genes Chromosomes Cancer. 2019 Mar. 12, Gallogly et Lazarus, J Blood Med. 2016 Apr. 19; 7:73-83; Pitoia et Jerkovich, Drug Des Devel Ther. 2016 Mar. 11; 10:1119-31), the disclosure of which being incorporated herein by reference. Patent applications also disclose XX kinase inhibitors, for instance and non-exhaustively WO19034538, WO17148440, WO15056683, WO13170671, WO13124869, WO13142382, WO13157540, WO11086085, WO09095399, WO09143389, WO08111441, WO08046802, WO06020145, WO06106437, WO06135719, the disclosure of which being incorporated herein by reference. Specific examples of FLT3 kinase inhibitors are disclosed in the following table.

The kinase inhibitors may target IGF1R (Insulin-like growth factor 1 receptor also known as Insulin-like growth factor I receptor (IGF-I receptor) or CD antigen: CD221; UniprotKB—P08069 or C9J5X1). The IGF1R kinase inhibitors are well-known. For instance, reviews are published disclosing such IGF1R kinase inhibitors (Qu et al, Oncotarget. 2017 Apr. 25; 8(17):29501-29518; Chen et al, Curr Top Med Chem. 2017 Nov. 20; 17(28):3099-3130), the disclosure of which being incorporated herein by reference. Patent applications also disclose IGF1R kinase inhibitors, for instance and non-exhaustively WO16082713, WO08076415, WO08000922, WO08076143, WO07121279, WO07083017, WO07075554, WO06080450, WO05095399, WO05097800, WO05037836, WO02092599, the disclosure of which being incorporated herein by reference. Specific examples of IGF1R kinase inhibitors are disclosed in the following table.

The kinase inhibitors may target c-Met (Hepatocyte growth factor receptor, also known as HGF/SF receptor, Proto-oncogene c-Met, Scatter factor receptor or Tyrosine-protein kinase Met; UniprotKB—P08581). The c-Met kinase inhibitors are well-known. For instance, reviews are published disclosing such c-Met kinase inhibitors (Zhang et al, Expert Opin Ther Pat. 2019 January; 29(1):25-41; Goździk-Spychalska et al, Curr Treat Options Oncol. 2014 December; 15(4):670-82; Bahrami et al, J Cell Physiol. 2017 October; 232(10):2657-2673; Zhang et al, Eur J Med Chem. 2016 Jan. 27; 108:495-504; Qi et al, World J Gastroenterol. 2015 May 14; 21(18):5445-53), the disclosure of which being incorporated herein by reference. Patent applications also disclose c-Met kinase inhibitors, for instance and non-exhaustively WO18153293, WO18187355, WO14000713, WO14032498, WO14067417, WO14180182, WO1307089, WO13107285, WO13149581, WO12006960, WO12015677, WO12034055, WO12048258, WO12075683, WO11039527, WO11079142, WO11121223, WO11143646, WO11149878, WO10007317, WO10007316, WO10007318, WO10019899, WO10059668, WO10089508, WO10089509, WO09143389, WO09143211, WO09056692, WO09093049, WO09068955, WO13013308, WO08023698, WO08008310, WO08102870, WO07036630, WO07066185, WO07023768, WO07002254, WO07002258, WO07111904, WO06104161, WO05082854, WO05082855, WO0160814 the disclosure of which being incorporated herein by reference. Specific examples of c-Met kinase inhibitors are disclosed in the following table.

The kinase inhibitors may target JAK (Tyrosine-protein kinase JAK2, also known as Janus kinase 2; UniprotKB—O60674). The JAK kinase inhibitors are well-known. For instance, reviews are published disclosing such JAK kinase inhibitors (He et al, Expert Opin Ther Pat. 2019 February; 29(2):137-149; Hobbs et al, Hematol Oncol Clin North Am. 2017 August; 31(4):613-626; Senkevitch et Durum, Cytokine. 2017 October; 98:33-41; Leroy et Constantinescu, Leukemia. 2017 May; 31(5):1023-1038; Jin et al, Pathol Oncol Res. 2019 Jan. 31), the disclosure of which being incorporated herein by reference. Patent applications also disclose JAK kinase inhibitors, for instance and non-exhaustively WO19034153, WO18215389, WO18215390, WO18204238, WO17006968, WO17079205, WO17091544, WO17097224, WO17129116, WO17140254, WO17215630, WO16027195, WO16032209, WO16116025, WO16173484, WO16191524, WO16192563, WO15174376, WO15039612, WO14111037, WO14123167, WO14146492, WO14186706, WO13091539, WO13188184, WO11076419, WO10085597, WO10051549, WO10083283, WO10135621, WO10142752, WO10149769, WO11003065, WO09132202, WO09143389, WO09062258, WO09114512, WO09145856, WO09155565, WO09155551, WO08047831, WO08109943, WO08116139, WO08157207, WO07070514, WO07084557, WO07117494, WO07007919, WO06034116, WO06056399, WO06069080, WO05095400, WO04058753, WO04041789, WO04041814, WO04041810, WO03101989, WO0152892, the disclosure of which being incorporated herein by reference. Specific examples of JAK kinase inhibitors are disclosed in the following table.

The kinase inhibitors may target PDGFR (Platelet-derived growth factor receptor, also known as Platelet-derived growth factor receptor, CD140 antigen-like family member; UniprotKB—P16234 (PGFRA) P09619 (PGFRB)). The PDGFR kinase inhibitors are well-known. For instance, reviews are published disclosing such PDGFR kinase inhibitors (Roskoski, Pharmacol Res. 2018 March; 129:65-83; Andrick et Gandhi, Ann Pharmacother. 2017 December; 51(12):1090-1098; Khalique et Banerjee, Expert Opin Investig Drugs. 2017 September; 26(9):1073-1081; Miyamoto et al, Jpn J Clin Oncol. 2018 Jun. 1; 48(6):503-513; Gallogly et Lazarus, J Blood Med. 2016 Apr. 19; 7:73-83; Pitoia et Jerkovich, Drug Des Devel Ther. 2016 Mar. 11; 10:1119-31; Chen et Chen, Drug Des Devel Ther. 2015 Feb. 9; 9:773-9), the disclosure of which being incorporated herein by reference. Patent applications also disclose PDGFR kinase inhibitors, for instance and non-exhaustively WO11119894, WO08016192, WO07004749, WO03077892, WO03077892, WO0164200, WO0125238, WO0172711, WO0172758, WO9957117, and WO9928304, the disclosure of which being incorporated herein by reference. Specific examples of PDGFR kinase inhibitors are disclosed in the following table.

The kinase inhibitors may target RET (Proto-oncogene tyrosine-protein kinase receptor Ret, also known as Cadherin family member 12 or Proto-oncogene c-Ret; UniprotKB—P07949). The RET kinase inhibitors are well-known. For instance, reviews are published disclosing such RET kinase inhibitors (Roskoski et Sadeghi-Nejad, Pharmacol Res. 2018 February; 128:1-17; Zschäbitz et Grüllich; Recent Results Cancer Res. 2018; 211:187-198; Grüllich, Recent Results Cancer Res. 2018; 211:67-75; Pitoia et Jerkovich, Drug Des Devel Ther. 2016 Mar. 11; 10:1119-31), the disclosure of which being incorporated herein by reference. Patent applications also disclose RET kinase inhibitors, for instance and non-exhaustively WO18071454, WO18136663, WO18136661, WO18071447, WO18060714, WO18022761, WO18017983, WO17146116, WO17161269, WO17146116, WO17043550, WO17011776, WO17026718, WO14050781, WO07136103, WO06130673, the disclosure of which being incorporated herein by reference. Specific examples of RET kinase inhibitors are disclosed in the following table.

The kinase inhibitors may target AXL (Tyrosine-protein kinase receptor UFO, also known as AXL oncogene; UniprotKB—P30530). The AXL kinase inhibitors are well-known. For instance, reviews are published disclosing such AXL kinase inhibitors (Myers et al, J Med Chem. 2016 Apr. 28; 59(8):3593-608; Grüllich, Recent Results Cancer Res. 2018; 211:67-75), the disclosure of which being incorporated herein by reference. Patent applications also disclose AXL kinase inhibitors, for instance and non-exhaustively WO18121228, WO17059280, WO17028797, WO16166250, WO16104617, WO16097918, WO16006706, WO15143692, WO15119122, WO15100117, WO15068767, WO15017607, WO15012298, WO13115280, WO13074633, WO12135800, WO12028332, WO10090764, WO10083465, WO10005876, WO10005879, WO09127417, WO09054864, WO08128072, WO08098139, WO08083353, WO08083357, WO08083354, WO08083356, WO08083367, WO08080134, WO08045978, WO07030680, the disclosure of which being incorporated herein by reference. Specific examples of AXL kinase inhibitors are disclosed in the following table.

The kinase inhibitors may target c-KIT (Mast/stem cell growth factor receptor Kit, also known as Piebald trait protein (PBT), Proto-oncogene c-Kit, Tyrosine-protein kinase Kit or p145 c-kit; UniprotKB—P10721). The c-KIT kinase inhibitors are well-known. For instance, reviews are published disclosing such c-KIT kinase inhibitors (Abbaspour Babaei et al, Drug Des Devel Ther. 2016 Aug. 1; 10:2443-59, Zschäbitz et Grüllich; Recent Results Cancer Res. 2018; 211:187-198; Miyamoto et al, Jpn J Clin Oncol. 2018 Jun. 1; 48(6):503-513; Chen et al, Curr Top Med Chem. 2017 Nov. 20; 17(28):3099-3130; Gallogly et Lazarus, J Blood Med. 2016 Apr. 19; 7:73-83; Pitoia et Jerkovich, Drug Des Devel Ther. 2016 Mar. 11; 10:1119-31, Chen et Chen, Drug Des Devel Ther. 2015 Feb. 9; 9:773-9), the disclosure of which being incorporated herein by reference. Patent applications also disclose c-KIT kinase inhibitors, for instance and non-exhaustively WO19034128, WO18112136, WO18112140, WO17167182, WO17121444, WO14202763, WO13033116, WO13033203, WO13033167, WO13033070, WO13014170, WO09105712, WO08011080, WO08005877, WO07124369, WO07092403, WO07038669, WO07026251, WO06106437, WO06135719, WO06060381, WO05073225, WO05021531, WO05021537, WO05021544, WO04080462, WO04014903, WO03035049, WO03002114, WO03003006, WO03004006, the disclosure of which being incorporated herein by reference. Specific examples of c-KIT kinase inhibitors are disclosed in the following table.

The kinase inhibitors may target Trk (Tropomyosin receptor kinase, also known as high affinity nerve growth factor receptor, neurotrophic tyrosine kinase receptor, or TRK-transforming tyrosine kinase protein; UniprotKB—P04629 (Trk1), Q16620 (Trk2), Q16288 (Trk3)). The Trk kinase inhibitors are well-known. For instance, reviews are published disclosing such Trk kinase inhibitors (Bhangoo et Sigal, Curr Oncol Rep. 2019 Feb. 4; 21(2):14, Pacenta et Macy, Drug Des Devel Ther. 2018 Oct. 23; 12:3549-3561; Cocco et al, Nat Rev Clin Oncol. 2018 December; 15(12):731-747; Lange et Lo, Cancers (Basel). 2018 Apr. 4; 10(4); Rolfo et al, Expert Opin Investig Drugs. 2015; 24(11):1493-500), the disclosure of which being incorporated herein by reference. Patent applications also disclose Trk kinase inhibitors, for instance and non-exhaustively WO18199166, WO18079759, WO17135399, WO17087778, WO17006953, WO16164286, WO16161572, WO16116900, WO16036796, WO16021629, WO15200341, WO15175788, WO15143653, WO15148350, WO15148344, WO15143654, WO15148373, WO15148354, WO15143652, WO15089139, WO15039334, WO15042085, WO15039333, WO15017533, WO14129431, WO14105958, WO14078417, WO14078408, WO14078378, WO14078372, WO14078331, WO14078328, WO14078325, WO14078322, WO14078323, WO13183578, WO13176970, WO13161919, WO13088257, WO13088256, WO13009582, WO12158413, WO12137089 WO12116217, WO12034091, WO12037155, WO11006074, WO10048314, WO10033941, WO09054468, WO08135785, WO07123269, WO06135719, WO06123113, WO06087538, WO06087530, WO06082392, WO05049033, WO03027111, the disclosure of which being incorporated herein by reference. Specific examples of Trk kinase inhibitors are disclosed in the following table.

The kinase inhibitors may target ROS1 (Proto-oncogene tyrosine-protein kinase ROS, also known as Proto-oncogene c-Ros, Proto-oncogene c-Ros-1, Receptor tyrosine kinase c-ros oncogene 1 and c-Ros receptor tyrosine kinase; UniprotKB—P08922). The ROS1 kinase inhibitors are well-known. For instance, reviews are published disclosing such ROS1 kinase inhibitors (Lin et Shaw, J Thorac Oncol. 2017 November; 12(11):1611-1625; Facchinetti et al, Cancer Treat Rev. 2017 April; 55:83-95; Rolfo et al, Expert Opin Investig Drugs. 2015; 24(11):1493-500, Yang et Gong, Expert Rev Clin Pharmacol. 2019 March; 12(3):173-178, Liu et al, Ther Clin Risk Manag. 2018 Jul. 20; 14:1247-1252; Sgambato et al, Expert Rev Anticancer Ther. 2018 January; 18(1):71-80), the disclosure of which being incorporated herein by reference. Patent applications also disclose ROS1 kinase inhibitors, for instance and non-exhaustively WO13183578, WO13180183, WO13158859, WO12037155, WO12005299, WO14141129, WO15144801, WO15144799, WO18170381, the disclosure of which being incorporated herein by reference. Specific examples of ROS1 kinase inhibitors are disclosed in the following table.

The kinase inhibitors may target BTK (Tyrosine-protein kinase BTK, also known as Agammaglobulinemia tyrosine kinase (ATK), B-cell progenitor kinase (BPK) and Bruton tyrosine kinase; UniprotKB—Q06187). The BTK kinase inhibitors are well-known. For instance, reviews are published disclosing such BTK kinase inhibitors (Kim H O, Arch Pharm Res. 2019 February; 42(2):171-181; Liang et al, Eur J Med Chem. 2018 May 10; 151:315-326, Aw et Brown, Drugs Aging. 2017 July; 34(7):509-527; Wu et al, Oncotarget. 2017 Jan. 24; 8(4):7201-7207, Wu et al, J Hematol Oncol. 2016 Sep. 2; 9(1):80), the disclosure of which being incorporated herein by reference. Patent applications also disclose BTK kinase inhibitors, for instance and non-exhaustively WO18002958, WO18001331, WO18009017, WO18035080, WO18088780, WO18090792, WO18095398, WO18133151, WO18145525, A1WO18154131, WO18175512, A1WO18192536, WO18192532, WO18196757, WO18208132, WO18233655, WO19034009, WO17007987, WO17046604, WO17066014, WO17077507, WO17123695, WO17127371, WO17128917, WO17190048, WO17106429, WO16019233, WO16057500, WO16065222, WO16066726, WO16106628, WO16106626, WO16106629, WO16109215, WO16106627, WO16106623, WO16106624, WO16106652, WO16112637, WO16161571, WO16161570, WO16196776, WO16196840, WO16192074, WO16210165, WO16109220, WO15017502, WO15002894, WO15022926, WO15048689, WO15048662, WO15061247, WO15084998, WO15095102, WO15095099, WO15116485, WO15169233, WO15165279, WO15132799, WO15039612, WO14104757, WO14113932, WO14114185, WO14113942, WO14116504, WO14130693, WO14164558, WO14151620, WO14152114, WO14161799, WO14187319, WO14210255, WO14005217, WO14025976, WO14039899, WO14055928, WO14055934, WO14068527, WO14078578, WO14082598, WO14082598, WO13067264, WO13081016, WO13102059, WO13116382, WO13148603, WO13152135, WO13185084, WO13067277, WO13067274, WO13059738, WO13010869, WO13010380, WO13010868, WO12170976, WO12135801, WO12021444, WO11153514, WO11152351, WO11029043, WO11029046, WO10126960, WO10056875, WO10009342, WO09156284, WO09098144, WO09053269, WO08121742, WO08039218, WO9954286, the disclosure of which being incorporated herein by reference. Specific examples of BTK kinase inhibitors are disclosed in the following table.

The kinase inhibitors may target Syk (Tyrosine-protein kinase SYK, also known as Spleen tyrosine kinase, p72-Syk; UniprotKB—P43405). The Syk kinase inhibitors are well-known. For instance, reviews are published disclosing such Syk kinase inhibitors (Bartaula-Brevik et al, Expert Opin Investig Drugs. 2018 April; 27(4):377-387; Liu et Mamorska-Dyga, J Hematol Oncol. 2017; 10: 145, Geahlen, Trends Pharmacol Sci. 2014 August; 35(8):414-22; Norman Expert Opin Ther Pat. 2014 May; 24(5):573-95), the disclosure of which being incorporated herein by reference. Patent applications also disclose Syk kinase inhibitors, for instance and non-exhaustively WO19034153, WO18053189, WO18053190, WO18108083, WO18228475, WO17046302, WO16010809, WO15138273, WO15140051, WO15140054, WO15140055, WO15144614, WO15017610, WO15061369, WO15094997, WO15095444, WO15095445, WO15100217, WO14051654, WO14048065, WO14060371, WO14064134, WO14074422, WO14086032, WO14093191, WO14100314, WO14176210, WO14176216, WO14023385, WO14027300, WO14031438, WO14029732, WO14045029, WO13192125, WO13192128, WO13192098, WO13192088, WO13047813, WO13052391, WO13052394, WO13052393, WO13064445, WO13099041, WO13104573, WO13104575, WO13109882, WO13124026, WO13126132, WO13124025, WO12002577 WO12025187 WO12025186, WO12061418, WO12123311, WO12123312, WO12130780, WO12151137, WO12154519, WO12154520, WO12154518, WO12167423, WO12167733, WO11086085, WO11014795, WO11014515, WO11075515, WO11075560, WO11079051, WO11092128, WO11112995, WO11117160, WO11134971, WO11144584, WO11144585, WO10068257, WO10068258, WO10097248, WO10147898, WO09131687, WO09136995, WO09145856, WO09031011, WO08033798, WO07129226, WO07042298, WO07042299, WO07028445, WO07009681, WO07009681, WO07085540, WO06093247, WO05033316, WO05026158, WO03063794, WO03057695, WO0183485, WO0147922, WO0109134, WO0075113, the disclosure of which being incorporated herein by reference. Specific examples of Syk kinase inhibitors are disclosed in the following table.

In a very specific aspect, the kinase inhibitor can be selected in the following table:

| Target | Type | Drug |
|---|---|---|
| EGFR | Tyrosine | gefitinib, erlotinib, lapatinib, |
| | | vandetanib, afatinib, osimertinib, |
| | | neratinib, dacomitinib, brigatinib, |
| | | canertinib, naquotinib, nazartinib, |
| | | pelitinib, rociletinib, icotinib, AZD3759, |
| | | AZ5104 (CAS No 1421373-98-9), |
| | | poziotinib, WZ4002 |
| ALK | Tyrosine | Crizotinib, entrectinib, ceritinib, |
| | | alectinib, brigatinib, lorlatinib, |
| | | TSR-011, CEP-37440, ensartinib |
| B-Raf | Serine/ | Vemurafenib, dabrafenib, regorafenib, |
| | threonine | PLX4720 |
| MEK1/2 | Dual | Cobimetinib, Trametinib, Binimetinib, |
| | specificity | Selumetinib, PD-325901, CI-1040, |
| | | PD035901, U0126, TAK-733 |
| FGFR | Tyrosine | Lenvatinib (FGFR1/2/3/4); Debio-1347 |
| family | | and dovitinib (FGFR1/2/3); |
| including | | BLU9931 (FGFR4); regorafenib |
| FGFR1, | | |
| FGFR2, | | |
| FGFR3 | | |
| and | | |
| FGFR4 | | |
| FLT3 | Tyrosine | Sorafenib, sunitinib, lestaurtinib, |
| | | tandutinib, quizartinib, crenolanib, |
| | | gilteritinib, ponatinib, ibrutinib |
| IGF1R | Tyrosine | Linsitinib, NVP-AEW541, BMS-536924, |
| | | AG-1024, GSK1838705A, |
| | | BMS-754807, PQ 401, ZD3463, NT157, |
| | | Picropodophyllin (PPP) |
| c-Met | Tyrosine | Tivantinib, JNJ-38877605, PF-04217903, |
| | | foretinib (GSK1363089), Merestinib |
| JAK | Tyrosine | Ruxolitinib, tofacitinib, oclacitinib, |
| | | baricitinib, filgotinib, cerdulatinib, |
| | | gandotinib, lestaurtinib, momelotinib, |
| | | pacritinib, PF-04965842, upadacitinib, |
| | | peficitinib, fedratinib |
| PDGFR | Tyrosine | imatinib, regorafenib, sunitinib, |
| α/β | | sorafenib, pazopanib, Telatinib, |
| | | bosutinib, nilotinib, ponatinib, lenvatinib |
| RET | Tyrosine | cabozantinib, vandetanib, lenvatinib |
| AXL | Tyrosine | Bemcentinib, amuvatinib, bosutinib, |
| | | cabozantinib, foretinib, gilteritinib |
| | | (ASP2215), glesatinib (MGCD 265), |
| | | SGI-7079 |
| TrkA, | Tyrosine | Larotrectinib, entrectinib, RXDX-102, |
| TrkB, TrkC | | altiratinib, LOXO-195, sitravatinib |
| ROS1 | Tyrosine | crizotinib, entrectinib, lorlatinib, ceritinib, |
| | | cabozantinib, TPX-0005, DS-6051b |
| BTK | Tyrosine | Ibrutinib, Acalabrutinib, GS-4059, |
| | | spebrutinib, BGB-3111, HM7122 |
| Syk | Tyrosine | fostamatinib, entospletinib, cerdulatinib, |
| | | TAK-659 |

The treatment with a kinase inhibitor can also be a combination of several kinase inhibitors which target the same kinase or different kinases. For instance, a treatment comprising several kinase inhibitors targeting different kinases can be a combination of a B-raf kinase inhibitor and a MEK kinase inhibitor, preferably a B-raf kinase inhibitor selected from the group consisting of Vemurafenib, dabrafenib, regorafenib and PLX4720 and a MEK kinase inhibitor selected from the group consisting of cobimetinib, trametinib, binimetinib, selumetinib, PD-325901, CI-1040, PD035901, U0126 and TAK-733, such as a combination of vemurafenib and trametinib. Alternatively, a kinase inhibitor may target different kinases.

In a particular aspect, the kinase inhibitor is an EGFR inhibitor. For instance, it can be selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS NO 1421373-98-9), poziotinib, WZ4002, more preferably erlotinib.

Cancers or Tumors to be Treated

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma.

Various cancers are also encompassed by the scope of the invention, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testis, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma (including cutaneous or peripheral T-cell lymphoma), Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatocarcinoma, breast cancer, colon carcinoma, and head and neck cancer, retinoblastoma, gastric cancer, germ cell tumor, bone cancer, bone tumors, adult malignant fibrous histiocytoma of bone; childhood malignant fibrous histiocytoma of bone, sarcoma, pediatric sarcoma; myelodysplastic syndromes; neuroblastoma; testicular germ cell tumor, intraocular melanoma, myelodysplastic syndromes; myelodysplastic/myeloproliferative diseases, synovial sarcoma.

In a preferred embodiment of the present invention, the cancer is a solid tumor. For instance, the cancer may be sarcoma and osteosarcoma such as Kaposi sarcome, AIDS-related Kaposi sarcoma, melanoma, in particular uveal melanoma, and cancers of the head and neck, kidney, ovary, pancreas, prostate, thyroid, lung, esophagus, breast in particular triple negative breast cancer (TNBC), bladder, colorectum, liver and biliary tract, uterine, appendix, and cervix, testicular cancer, gastrointestinal cancers and endometrial and peritoneal cancers. Preferably, the cancer may be sarcoma, melanoma, in particular uveal melanoma, and cancers of the head and neck, kidney, ovary, pancreas, prostate, thyroid, lung, esophagus, breast in particular (TNBC), bladder, colorectum, liver, cervix, and endometrial and peritoneal cancers.

In a particular aspect, the cancer can be selected from the group consisting of leukemia, lymphoma, sarcoma, melanoma, and cancers of the head and neck, kidney, ovary, pancreas, prostate, thyroid, lung, esophagus, breast, bladder, brain, colorectum, liver, and cervix.

In another aspect, the cancer can be selected from the group consisting of lung cancer, in particular non-small cell lung cancer, leukemia, in particular acute myeloid leukemia, chronic lymphocytic leukemia, lymphoma, in particular peripheral T-cell lymphoma, chronic myelogenous leukemia, squamous cell carcinoma of the head and neck, advanced melanoma with BRAF mutation, colorectal cancer, gastrointestinal stromal tumor, breast cancer, in particular HER2⁺ breast cancer, thyroid cancer, in particular advanced medullary thyroid cancer, kidney cancer, in particular renal cell carcinoma, prostate cancer, glioma, pancreatic cancer, in particular pancreatic neuroendocrine cancer, multiple myeloma, and liver cancer, in particular hepatocellular carcinoma.

For instance, if the kinase inhibitor is an EGFR inhibitor, the cancer is preferably selected from the group consisting of lung cancer, in particular non-small cell lung cancer, pancreatic cancer, breast cancer, in particular early breast cancer, thyroid cancer, in particular medullary thyroid cancer, colorectal cancer, in particular metastatic or advanced colorectal cancer, squamous cell carcinoma of the head and neck and glioma. In a particular aspect, if the kinase inhibitor is an EGFR inhibitor, the cancer is preferably lung cancer, in particular non-small cell lung cancer. If the kinase inhibitor is an ALK inhibitor, the cancer is preferably lung cancer, in particular non-small cell lung cancer. If the kinase inhibitor is a B-Raf inhibitor, the cancer is preferably selected from the group consisting of melanoma, lung cancer, colorectal cancer and gastro-intestinal stromal cancer, in particular an advanced melanoma with BRAF mutation. If the kinase inhibitor is an MEK inhibitor, the cancer is preferably melanoma or lung cancer, in particular an advanced melanoma with BRAF mutation. If the kinase inhibitor is a FGFR inhibitor, the cancer is preferably selected from the group consisting of thyroid carcinoma, colorectal cancer and gastro-intestinal stromal cancer. If the kinase inhibitor is a FLT3 inhibitor, the cancer is preferably selected from the group consisting of kidney cancer, pancreatic cancer, especially pancreatic neuroendocrine tumor, gastro-intestinal stromal cancer, multiple myeloma, prostate cancer, leukemia such as acute myeloid leukemia and chronic lymphocytic leukemia, and lymphoma. If the kinase inhibitor is a JAK inhibitor, the cancer is preferably selected from the group consisting of lymphoma, especially peripheral T-cell lymphoma, myeloproliferative neoplasms, multiple myeloma, pancreatic cancer, and prostate cancer. If the kinase inhibitor is a PDGFR inhibitor, the cancer is preferably selected from the group consisting of leukemia such as Philadelphia chromosome-positive chronic myeloid leukemia, gastro-intestinal stromal cancer, myelodysplastic and myeloproliferative syndromes, colorectal cancer, kidney cancer, pancreatic cancer, in particular pancreatic neuroendocrine tumor, liver cancer, breast cancer, and thyroid carcinoma. If the kinase inhibitor is a RET inhibitor, the cancer is preferably kidney cancer or thyroid cancer such as medullary thyroid cancer. If the kinase inhibitor is an AXL inhibitor, the cancer is preferably selected from the group consisting of leukemia, in particular acute leukemia such as acute myeloid leukemia or Philadelphia chromosome-positive chronic myeloid leukemia, kidney cancer, and lung cancer such as NSCLC. If the kinase inhibitor is a Trk inhibitor, the cancer is preferably a metastatic solid cancer. If the kinase inhibitor is a ROS1 inhibitor, the cancer is preferably selected from the group consisting of lung cancer such as NSCLC and kidney cancer. If the kinase inhibitor is a BTK inhibitor, the cancer is preferably selected from the group consisting of B cell cancers such as chronic lymphocytic leukemia (CLL) and non-Hodgkin lymphoma. If the kinase inhibitor is a Syk inhibitor, the cancer is preferably lymphoma, especially peripheral T-cell lymphoma.

If the kinase inhibitor treatment is a combination of B-Raf kinase inhibitor and MEK1/2 kinase inhibitor, such as a combination of vemurafenib and trametinib, the cancer to be treated could be a melanoma, more particularly an advanced melanoma with BRAF mutation.

In a particular aspect, the present invention discloses a pharmaceutical composition, a combination or a kit comprising a Dbait molecule and several kinase inhibitors, in particular a combination of B-Raf and MEK1/2 inhibitors. In a particular embodiment, the combination could be a combination of vemurafenib and trametinib.

Therefore, the present invention discloses a pharmaceutical composition, a combination or a kit comprising a Dbait molecule as defined herein, and vemurafenib and trametinib for use for treating melanoma, more particularly an advanced melanoma with BRAF mutation.

The pharmaceutical compositions and the products, kits, combinations or combined preparations described in the invention may be useful for inhibiting the growth of solid tumors, decreasing the tumor volume, preventing the metastatic spread of tumors and the growth or development of micrometastases, preventing the tumor recurrence and preventing the tumor relapse. The pharmaceutical compositions and the products, kits, combinations, or combined preparations described in the invention are in particular suitable for the treatment of poor prognosis patients or of radio- or chemo-resistant tumors. In a particular embodiment, the cancer is a high-grade or advanced cancer or is a metastatic cancer.

Regimen, Dosages and Administration Routes

The effective dosage of each of the combination partners employed in the combined preparation of the invention may vary depending on the particular compound or pharmaceutical composition employed, the mode of administration, the condition being treated, the severity of the condition being treated. Thus, the dosage regimen of the combined preparation of the invention is selected in accordance with a variety of factors including the route of administration and the patient status. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The pharmacological activity of a combination of the invention may, for example, be demonstrated in a clinical study or more preferably in a test procedure. Suitable clinical studies are, for example, open label non-randomized, dose escalation studies in patients with advanced tumors. Such studies can prove the synergism of the active ingredients of the combination of the invention. The beneficial effects on proliferative diseases can be determined directly through the results of these studies or by changes in the study design which are known as such to a person skilled in the art. Such studies are, in particular, suitable to compare the effects of a monotherapy using the active ingredients and a combination of the invention. Preferably, the combination partner (a) is administered with a fixed dose and the dose of the combination partner (b) is escalated until the maximum tolerated dosage is reached. Alternatively, the combination partner (b) is administered with a fixed dose and the dose of the combination partner (a) is escalated until the maximum tolerated dosage is reached.

In some embodiments, “combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Preferably, the Dbait molecule and the kinase inhibitor are administered concomitantly or simultaneously.

The term “concomitantly” is used herein to refer to administration of two or more therapeutic agents, give in close enough temporal proximity where their individual therapeutic effects overlap in time. Accordingly, concurrent administration includes a dosing regimen when the administration of one or more agent(s) continues after discontinuing the administration of one or more other agent(s).

The Dbait molecule and the kinase inhibitor can have same or different administration regimen. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, or any combination thereof. For example, in one embodiment, the first agent can be administered prior to the second therapeutic agent, for e.g. 1 week. In another, the first agent can be administered prior to (for example 1 day prior) and then concomitant with the second therapeutic agent.

The Dbait molecule and the kinase inhibitor may be administered by the same route or by distinct routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Therapeutic agents may also be administered in alternation. The administration route could be oral, parenteral, intravenous, intratumoral, subcutaneous, intracranial, intraartery, topical, rectal, transdermal, intradermal, nasal, intramuscular, intraosseous, and the like.

The treatment may include one or several cycles, for instance two to ten cycles, in particular two, three, four or five cycles. The cycles may be continued or separated. For instance, each cycle is separated by a period of time of one to eight weeks, preferably three to four weeks.

Further aspects and advantages of the present invention will be described in the following examples, which should be regarded as illustrative and not limiting.

### EXAMPLES

### Example 1

Material and Methods

To demonstrate the specific effect of AsiDNA on persister cells, the inventors chose as model system two well-known epidermal growth factor receptor (EGFR)-addicted non-small cell lung cancer (NSCLC) cell lines: PC9 and HCC827.

EGFR T790 mutation is preexisting in PC9 parental cell line (Hata et al., Nat. Med. 2016). PC9-3 cell line is the result of a subcloning of PC9 without preexisting T790 mutation. HCC827 sc2 and sc3 are also the result of subcloning of HCC827 without preexisting T790 mutation. Thus, in PC9-3 and HCC827 sc2 cell lines, proliferation under Erlotinib treatment is due to adaptive mechanisms from persister cells.

Cell Culture

The human NSCLC cell lines, HCC827 cell line (CRL-2868, EGFR del E749-A750) and the PC9 cell line (EGFR del E746-A750) were kind gifts from Antonio Maraver (IRCM, Montpellier, France). Cell lines were cultured in RPMI 1640 medium containing 10% fetal bovine serum (FBS), and were maintained at 37° C. in a humidified chamber containing 5% CO2. Cell lines were authenticated by short tandem repeat (STR) analysis using PowerPlex 16 HS (Promega).

Cell Proliferation Assay

PC9 cells were seeded in 96 well plates 24 h before treatment at a density of 20000 cells/cm2. Cells were treated for 5 days at several doses of Erlotinib with or without AsiDNA at 1, 5 or 10 μM, and the relative number of viable cells was measured by incubating cells with the MTS reagent (CellTiter 96® AQueous One Solution Cell Proliferation Assay from Promega), as recommended by the manufacturer. Relative cell survival in the presence of drugs was normalized to the untreated cells after background corrections.

Drug Treatments, Persister AsiDNA Response

Cells were seeded in 6-well culture plates at appropriate densities and incubated 24 h at 37° C. before addition of Erlotinib (1 μM), or AsiDNA (1 μM, or 5 μm, or 10 μM) or combination of both drugs. Cells were treated for 21 days and control medium as well as drug-containing medium were replaced twice per week. Surviving cells were washed, PFA-fixed and stained with Crystal violet. Plates were scanned using ChemiDoc Imaging System (Bio-Rad) and percentage of surviving cells was quantified using Nikon NIS Elements Imaging Software.

Results

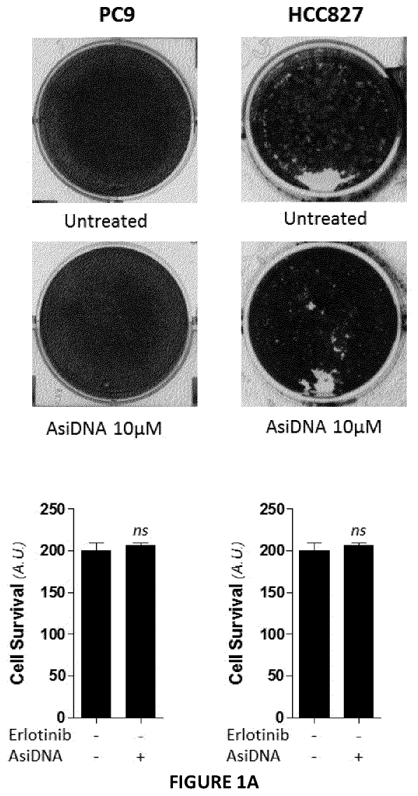

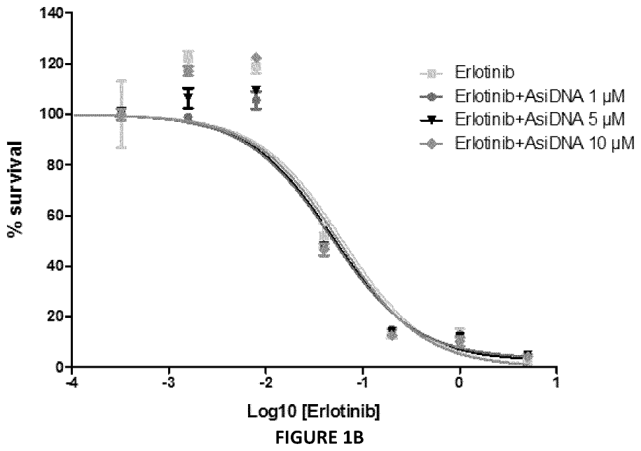

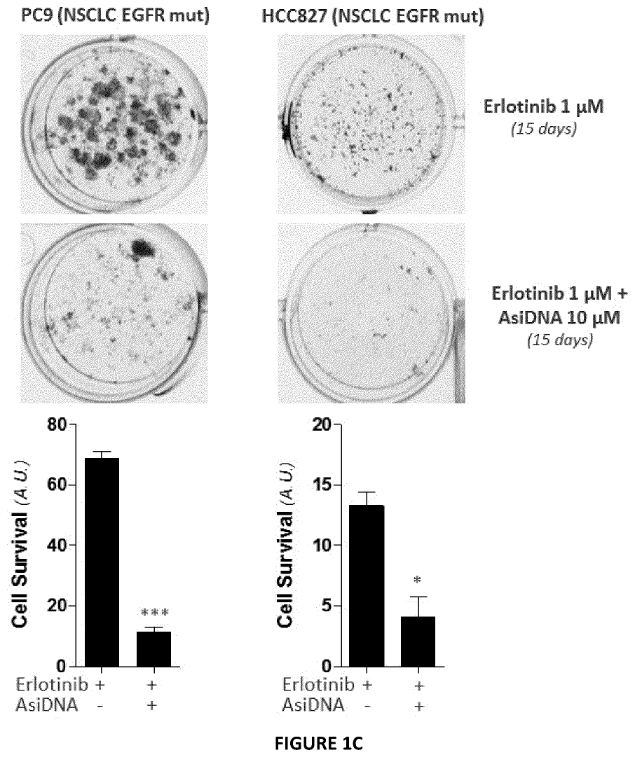

AsiDNA treatment alone did not affect cell survival (FIG. 1A). AsiDNA does not potentiate erlotinib-mediated cell death (FIG. 1B) but AsiDNA strongly decreased the proportion of emerging erlotinib-resistant clones lines (FIG. 1C) in the PC9-3 and the HCC827 sc2 cell lines, demonstrating an efficacy of AsiDNA against persister cell regrowth.

### Example 2

Material and Methods

Cell Culture

The human NSCLC cell line HCC827 (CRL-2868, EGFR del E749-A750) was obtained from the American Type Culture Collection (ATCC, Manassas, Va., USA). The human NSCLC cell PC9 (EGFR del E746-A750) was a kind gift from Antonio Maraver (IRCM, Montpellier). NSCLC cell lines were cultured in RPMI 1640 medium containing 10% fetal bovine serum (FBS), and were maintained at 37° C. in a humidified chamber containing 5% CO2. Cell lines were authenticated by short tandem repeat (STR) analysis using PowerPlex 16 HS (Promega).

As cell lines may harbor a pre-existing resistant subpopulation, all cell lines were subcloned (i.e. derived from a single cell and amplified without drug pressure in a limited number of passages) to specifically focus on the drug-tolerant state and the emergence of de novo resistance mechanisms.

For fluorescence monitoring, all cells were transduced with a GFP lentivirus (M01=2) and green fluorescent populations were sorted by FACS.

Drug Treatments, Measurement of Persister Survival

Cell lines were treated or not with Erlotinib (1 μM) with or without AsiDNA (10 μM) and survival curves (drug response and relapse) were monitored by fluorescence detection using a spectrofluorometer (Synergy 2, BioTek). Medium was changed twice a week, and fluorescence measurements were performed just after medium change.

Results

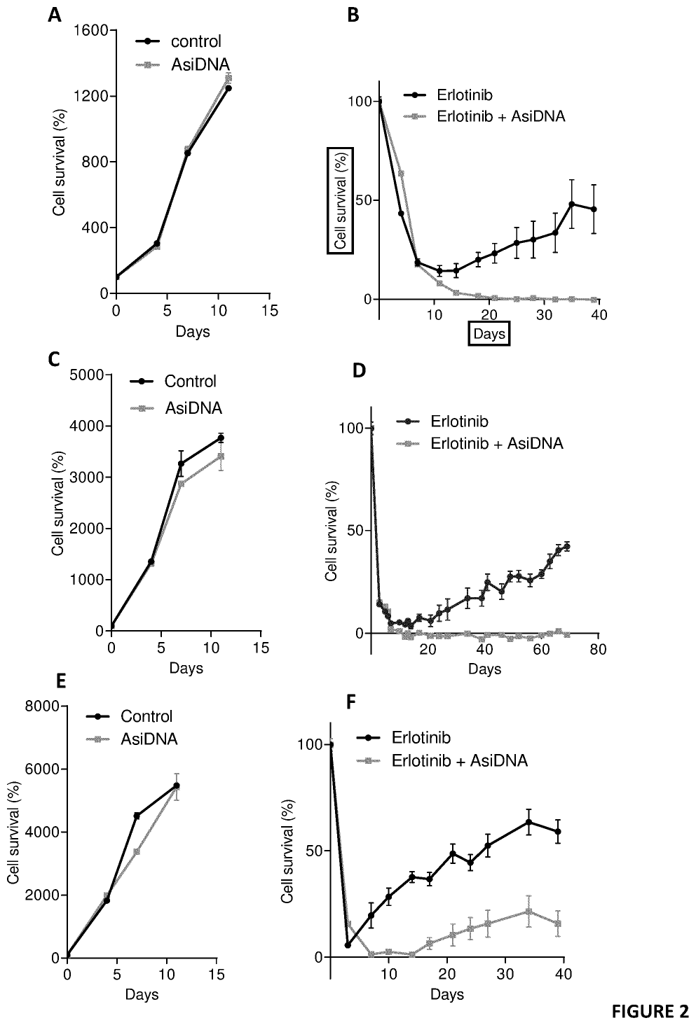

AsiDNA treatment alone did not affect cell survival (FIG. 2A-2C-2E). AsiDNA totally abrogated Erlotinib acquired resistance on the two subclones HCC827 sc2 (FIG. 2B) and PC9-3 (FIG. 2D) while it partially but significantly reduced resistance on PC9 parental cell line (FIG. 2F) further demonstrating the long term efficacy of AsiDNA on persister cells.

### Example 3

Cell Culture

The human NSCLC cell PC9 (EGFR del E746-A750) were a kind gift from Antonio Maraver (IRCM, Montpellier). NSCLC cell PC9 were cultured in RPMI 1640 medium containing 10% fetal bovine serum (FBS), and were maintained at 37° C. in a humidified chamber containing 5% CO2. Cell lines were authenticated by short tandem repeat (STR) analysis using PowerPlex 16 HS (Promega).

For fluorescence monitoring, all cells were transduced with a GFP lentivirus (M01=2) and green fluorescent populations were sorted by FACS.

Drug Treatments, Measurement of Persister Survival

PC9 cells were treated or not with Osimertinib (1 μM) with or without AsiDNA (10 μM) and survival curves (drug response and relapse) were monitored by fluorescence detection using a spectrofluorometer (Synergy 2, BioTek). Medium was changed twice a week, and fluorescence measurements were performed just after medium change.

Results

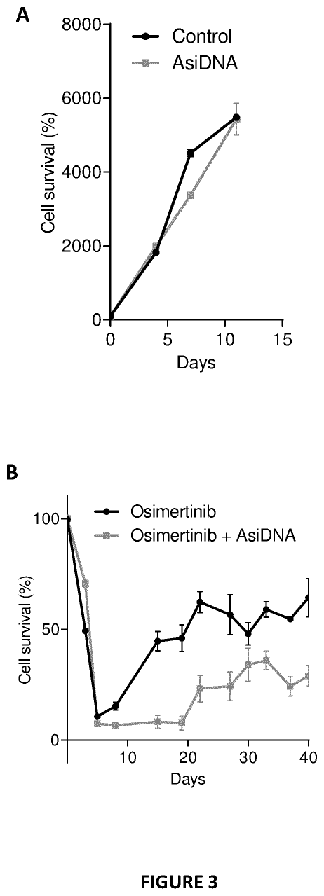

AsiDNA treatment alone did not affect cell survival (FIG. 3A). AsiDNA significantly reduced Osimertinib resistance on PC9 parental cell line (FIG. 3B). These results confirming the results obtained precedently with another TKi Erlotinib.

### Example 4

Material and Methods

Cell Culture

The human NSCL cancer cell line H3122 (NSCL cancer model expressing EML4-ALK) was a kind gift from Antonio Maraver (IRCM, Montpellier). NSCLC cell line H3122 was cultured in RPMI 1640 medium containing 10% fetal bovine serum (FBS), and were maintained at 37° C. in a humidified chamber containing 5% CO2. Cell lines were authenticated by short tandem repeat (STR) analysis using PowerPlex 16 HS (Promega).

For fluorescence monitoring, cells were transduced with a GFP lentivirus (M01=2) and green fluorescent populations were sorted by FACS.

Drug Treatments, Measurement of Persister Survival

Cell line was treated or not with Alectinib (2 μM) with or without AsiDNA (10 μM) and survival curves (drug response and relapse) were monitored by fluorescence detection using a spectrofluorometer (Synergy 2, BioTek). Medium was changed twice a week, and fluorescence measurements were performed just after medium change.

Results

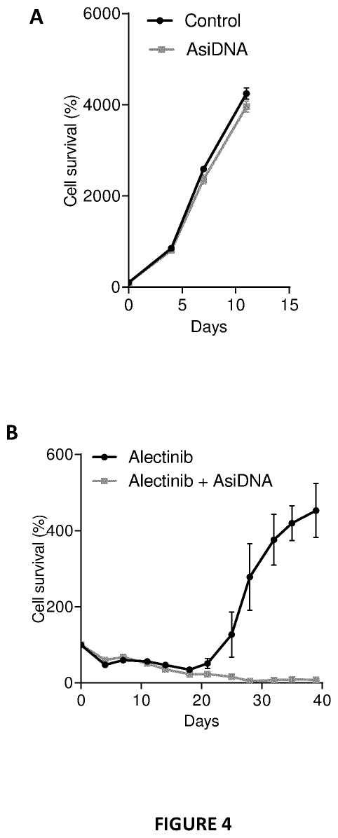

AsiDNA treatment alone did not affect cell survival (FIG. 4A). AsiDNA totally abrogated Alectinib acquired resistance (FIG. 4B) demonstrating the efficacy of AsiDNA on a general mechanism of resistance to TKi driven by drug tolerant cells. AsiDNA abrogated resistance to Alectinib on H3122 cells, confirming its cytotoxic activity on persister cells.

### Example 5

Material and Methods

Mouse Model

6-week old female NMRI nude mice (Crl:NMRI-Foxn1nu) were purchased from Charles River Laboratories, France. Animals were allowed to acclimate for at least 5 days before initiation of the study. All in vivo studies were conducted at CREFRE (INSERM 0006) with the approval of the Animal Care and Ethical Committee (#4181-2016040116494282). Animals were housed under controlled temperature and lighting (12/12 h light/dark cycle), fed with commercial animal feed and water ad libitum. All procedures involving animals and their care conformed to institutional guidelines for the use of animals in biomedical research.

PC9 Xenograft

PC9 cells were harvested, and 5×106 cells were implanted subcutaneously in the left flank of the NMRI nude mice.

Drug Treatments, Measurement of Tumor Volume

When the tumors reached an average of 250±50 mm3, the mice were randomly assigned to receive either vehicle, or 10 mg/kg Erlotinib, or 10 mg AsiDNA (10 mice/group). Erlotinib was administered once daily, 5 days/week, orally as a suspension using 0.5% hydroxypropyl methylcellulose (HPMC) with 0.1% Tween 80 as vehicle. AsiDNA was prepared in NaCl 0.9% solution, stored at −20° C. and warmed to 37° C. prior to administration. AsiDNA was administered alone or in combination with Erlotinib by intraperitoneal injections (10 mg/mice) at day 1, 2 and 3 of treatment, then once a week. Control vehicle treated mice received 0.5% HPMC with 0.1% Tween 80 administered orally. Mice were treated for 10 weeks and tumor volumes were determined twice a week from caliper measurements by using the formula V=(length×width2)/2.

Results

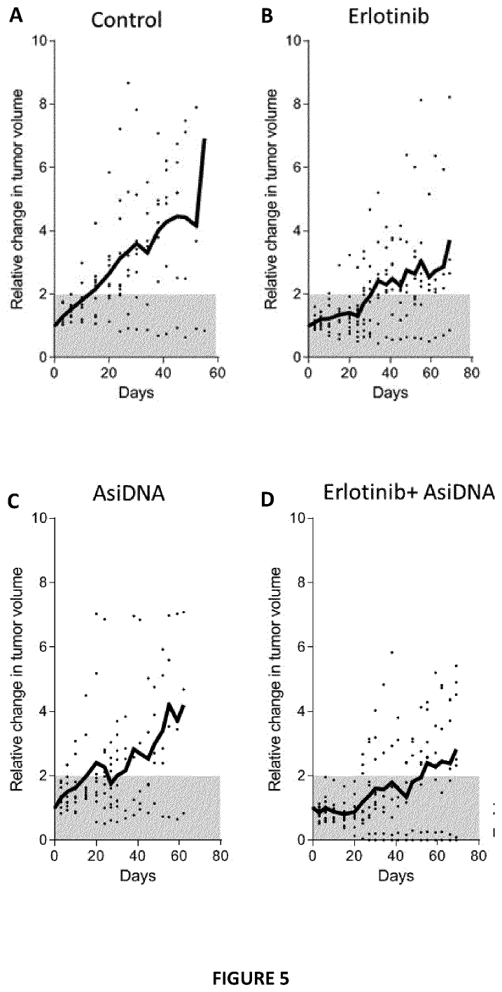

The treatment with Erlotinib alone is able only to transiently control the tumor growth like in clinical situation (FIG. 5B). Treatment with AsiDNA slightly reduced the tumor growth (FIG. 5C) while the combination of both drugs reduced significantly the tumor growth and induced two complete regressions (FIG. 5D) demonstrating in an in vivo setting the potential of AsiDNA to control EGFR-TKi acquired resistance.

1-16. (canceled)

17. A pharmaceutical composition, a combination, or a kit comprising a Dbait molecule and a protein kinase inhibitor.

18. The pharmaceutical composition, the combination or the kit according to claim 17, wherein the kinase inhibitor is an inhibitor targeting one or several targets selected from the group consisting of EGFR family, ALK, B-Raf, MEK, FGFR1, FGFR2, FGFR3, FGFR4, FLT3, IGF1R, c-Met, JAK family, PDGFR α and β, RET, AXL, c-KIT, TrkA, TrkB, TrkC, ROS1, BTK and Syk.

19. The pharmaceutical composition, the combination or the kit according to claim 17, wherein the Dbait molecule has at least one free end and a DNA double stranded portion of 20-200 bp with less than 60% sequence identity to any gene in a human genome.

20. The pharmaceutical composition, the combination or the kit according to claim 17, wherein the Dbait molecule has one of the following formulae: 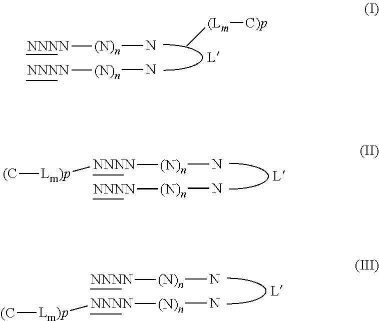
wherein N is a deoxynucleotide, n is an integer from 15 to 195, the underlined N refers to a nucleotide having or not a modified phosphodiester backbone, L′ is a linker, C is the molecule facilitating endocytosis selected from a lipophilic molecule or a ligand which targets cell receptor enabling receptor mediated endocytosis, L is a linker, m and p, independently, are an integer being 0 or 1.

21. The pharmaceutical composition, the combination or the kit according to claim 20, wherein the Dbait molecule has the following formula:

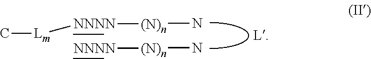

22. The pharmaceutical composition, the combination or the kit according to claim 17, wherein the Dbait molecule has the following formula:

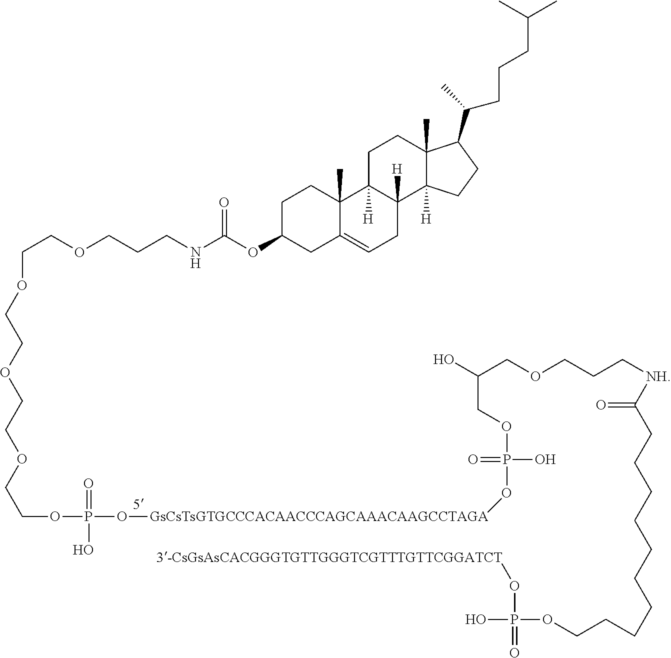

23. The pharmaceutical composition or the kit according to claim 17, wherein the kinase inhibitor is selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No. 1421373-98-9), poziotinib, WZ4002, Crizotinib, entrectinib, ceritinib, alectinib, lorlatinib, TSR-011, CEP-37440, ensartinib, Vemurafenib, dabrafenib, regorafenib, PLX4720, Cobimetinib, Trametinib, Binimetinib, Selumetinib, PD-325901, CI-1040, PD035901, U0126, TAK-733, Lenvatinib, Debio-1347, dovitinib, BLU9931, Sorafenib, sunitinib, lestaurtinib, tandutinib, quizartinib, crenolanib, gilteritinib, ponatinib, ibrutinib, Linsitinib, NVP-AEW541, BMS-536924, AG-1024, GSK1838705A, BMS-754807, PQ 401, ZD3463, NT157, Picropodophyllin (PPP), Tivantinib, JNJ-38877605, PF-04217903, foretinib (GSK 1363089), Merestinib, Ruxolitinib, tofacitinib, oclacitinib, baricitinib, filgotinib, cerdulatinib, gandotinib, momelotinib, pacritinib, PF-04965842, upadacitinib, peficitinib, fedratinib, imatinib, pazopanib, Telatinib, bosutinib, nilotinib, cabozantinib, Bemcentinib, amuvatinib, gilteritinib (ASP2215), glesatinib (MGCD 265), SGI-7079, Larotrectinib, RXDX-102, altiratinib, LOXO-195, sitravatinib, TPX-0005, DS-6051b, fostamatinib, entospletinib and TAK-659.

24. The pharmaceutical composition or the kit according to claim 17, wherein the kinase inhibitor is a tyrosine kinase inhibitor and is an inhibitor of a protein kinase selected from the group consisting of EGFR, ALK and B-Raf and is a tyrosine protein kinase inhibitor selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No. 1421373-98-9), poziotinib, WZ4002, Crizotinib, entrectinib, ceritinib, alectinib, lorlatinib, TSR-011, CEP-37440, ensartinib, Vemurafenib, dabrafenib, regorafenib and PLX4720.

25. The pharmaceutical composition, the combination or the kit according to claim 17, wherein the protein kinase inhibitor is an EGFR inhibitor selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No. 1421373-98-9), poziotinib and WZ4002.

26. The pharmaceutical composition, the combination or the kit according to claim 17, wherein the protein kinase inhibitor is an ALK inhibitor selected from the group consisting of crizotinib, entrectinib, ceritinib, alectinib, brigatinib, lorlatinib, TSR-011, CEP-37440 and ensartinib.

27. The pharmaceutical composition or the kit according to claim 22, wherein the kinase inhibitor is selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No. 1421373-98-9), poziotinib, WZ4002, Crizotinib, entrectinib, ceritinib, alectinib, lorlatinib, TSR-011, CEP-37440, ensartinib, Vemurafenib, dabrafenib, regorafenib, PLX4720, Cobimetinib, Trametinib, Binimetinib, Selumetinib, PD-325901, CI-1040, PD035901, U0126, TAK-733, Lenvatinib, Debio-1347, dovitinib, BLU9931, Sorafenib, sunitinib, lestaurtinib, tandutinib, quizartinib, crenolanib, gilteritinib, ponatinib, ibrutinib, Linsitinib, NVP-AEW541, BMS-536924, AG-1024, GSK1838705A, BMS-754807, PQ 401, ZD3463, NT157, Picropodophyllin (PPP), Tivantinib, JNJ-38877605, PF-04217903, foretinib (GSK 1363089), Merestinib, Ruxolitinib, tofacitinib, oclacitinib, baricitinib, filgotinib, cerdulatinib, gandotinib, momelotinib, pacritinib, PF-04965842, upadacitinib, peficitinib, fedratinib, imatinib, pazopanib, Telatinib, bosutinib, nilotinib, cabozantinib, Bemcentinib, amuvatinib, gilteritinib (ASP2215), glesatinib (MGCD 265), SGI-7079, Larotrectinib, RXDX-102, altiratinib, LOXO-195, sitravatinib, TPX-0005, DS-6051b, fostamatinib, entospletinib and TAK-659.

28. The pharmaceutical composition or the kit according to claim 22, wherein the kinase inhibitor is a tyrosine kinase inhibitor and is an inhibitor of a protein kinase selected from the group consisting of EGFR, ALK and B-Raf and is a tyrosine protein kinase inhibitor selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No. 1421373-98-9), poziotinib, WZ4002, Crizotinib, entrectinib, ceritinib, alectinib, lorlatinib, TSR-011, CEP-37440, ensartinib, Vemurafenib, dabrafenib, regorafenib and PLX4720.

29. The pharmaceutical composition, the combination or the kit according to claim 22, wherein the protein kinase inhibitor is an EGFR inhibitor selected from the group consisting of gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No. 1421373-98-9), poziotinib and WZ4002.

30. The pharmaceutical composition, the combination or the kit according to claim 22, wherein the protein kinase inhibitor is an ALK inhibitor selected from the group consisting of crizotinib, entrectinib, ceritinib, alectinib, brigatinib, lorlatinib, TSR-011, CEP-37440 and ensartinib.

31. A method of treating cancer comprising the administration of the combination or the kit according to claim 17 to a patient in need of treatment.

32. The method according to claim 31, said method delaying and/or preventing development of a cancer resistant to a kinase inhibitor in a patient.

33. The method according to claim 31, wherein the cancer is selected from the group consisting of leukemia, lymphoma, sarcoma, melanoma, cancers of the head and neck, kidney, ovary, pancreas, prostate, thyroid, lung, esophagus, breast, bladder, brain, colorectum, liver, and cervix.

34. The method according to claim 31, wherein the cancer is selected from the group consisting of lung cancer, non-small cell lung cancer, leukemia, acute myeloid leukemia, chronic lymphocytic leukemia, lymphoma, peripheral T-cell lymphoma, chronic myelogenous leukemia, squamous cell carcinoma of the head and neck, advanced melanoma with BRAF mutation, colorectal cancer, gastrointestinal stromal tumor, breast cancer, HER2⁺ breast cancer, thyroid cancer, advanced medullary thyroid cancer, kidney cancer, renal cell carcinoma, prostate cancer, glioma, pancreatic cancer, pancreatic neuroendocrine cancer, multiple myeloma, and liver cancer.

35. A method of reducing the number of cancer persister cells comprising the administration of a pharmaceutical composition, a combination, or a kit comprising a Dbait molecule and a protein kinase inhibitor according to claim 17 to a patient in need of treatment.
